(19) United States
(12) Patent Application Publication
(10) Pub. No.: US 2007/0065904 A1
(43) Pub. Date: Mar. 22, 2007

(54) Recombinant expression vectors for functional nav1.9 sodium channels

(76) Inventors: Matthew Gonda, Birmingham, AL (US); John D. Greenwood, Birmingham, AL (US)

Correspondence Address: CHOATE, HALL & STEWART LLP, TWO INTERNATIONAL PLACE, BOSTON, MA, 02110, US

(21) Appl. No.: 10/508,965

(22) PCT Filed: Mar. 20, 2003
(86) PCT No.: PCT/US03/08611

§ 371 Date: Apr. 11, 2005

(30) Foreign Application Priority Data
Mar. 20, 2002 (US) 60365550

(51) Int. Cl. C07H 21/04; C12N 15/09; C07K 14/705

(52) U.S. Cl. 435/69.1; 435/320.1; 435/325; 530/350; 536/23.5

(57) ABSTRACT

The present invention is drawn to the stable cloning and expression of Naᵥ1.9 sodium channel at the mRNA and protein levels, production of sodium channel currents characteristic of native currents in dorsal root ganglion neurons and to cell lines transiently or stably expressing recombinant Naᵥ1.9 sodium channel.

### RELATED APPLICATION DATA

This application claims the benefit of U.S. Provisional Application 60/365,550 (filed Mar. 20, 2002), which is herein incorporated by reference in its entirety.

### FIELD OF THE INVENTION

The present invention relates to expression vectors encoding a tetrodotoxin resistant sodium channel, the expression of recombinant sodium channel proteins and cell lines which transiently and stably express tetrodotoxin resistant sodium channels.

### BACKGROUND

Voltage-gated sodium channels are a class of specialized protein molecules that act as molecular batteries permitting excitable cells (neurons and muscle fibers) to produce and propagate electrical impulses. Voltage-gated sodium channels from rat brain are composed of three subunits, the pore-forming α subunit (260 kDa) and two auxiliary subunits, β1 (36 kDa) and β2 (33 kDa) that may modulate the properties of the α-subunit; the α subunit is sufficient to form a functional channel that generates a Na⁺ current flow across the membrane (Catterall (1993) Trends Neurosci. 16, 500-506; Isom et al. (1994) Neuron 12, 1183-1194). Nine distinct α subunits have been identified in vertebrates and are encoded by members of an expanding gene family (North (1995) Handbook of Receptors and Channels (pp. 73-100), CRC Press; Akopian et al. (1996) Nature 379, 257-262; Akopian et al. (1997) FEBS Letters 400, 183-187; Sangameswaran et al. (1996) J. Biol. Chem. 271, 5953-5956) and respective orthologues of a number of them have been cloned from various mammalian species including humans. Specific α subunits are expressed in a tissue- and developmentally-specific manner (Beckh et al. (1989) EMBO J. 8, 3611-3616; Mandel (1992) J. Membr. Biol. 125, 193-205). Aberrant expression patterns or mutations of voltage-gated sodium channel α-subunits underlie a number of human and animal disorders (Roden et al. (1997) Am. J. Physiol. 273, H511-525; Ptacek (1997) Neuromuscul. Disord. 7, 250-255; Cannon (1997) Neuromuscul. Disord. 7, 241-249; Cannon (1996) Trends Neurosci. 19, 3-10; Rizzo et al. (1996) European Neurology 36, 3-12).

Voltage-gated sodium channel α-subunits consist of four domains (D1-4) of varying internal homology but of similar predicted structure, connected by three intracellular loops (L1-3). The four domains fold to form a channel that opens to both the cytoplasm and the extracellular space via a pore. The pore opens and closes depending upon the physiological state of the cell membrane.

Each domain consists of six transmembrane segments (S1-6) that allow the protein to weave through the membrane with intra- and extracellular linkers. The S5-S6 segments of each of the four domains contain sequences that line the pore of the channel, and a highly conserved subset of amino acids that acts as a filter to selectively allow sodium ions to traverse the channel pore into the cytoplasm, thus generating a membrane potential. The amphiphatic S4 segment, in each of the four domains, rich in basic residues repeated every third amino acid, acts as a voltage sensor and undergoes a conformational change as a result of the change in the voltage difference across the cell membrane. This in turn triggers the conformational change of the protein to open its pore to the extracellular sodium ion gradient.

In most of the known voltage-gated sodium channel α-subunits the channels close and change into an inoperable state quickly (inactivate) within a few milliseconds after opening of the pore (activation); SNS-type channels, on the other hand, inactivate slowly and require a greater voltage change to activate. L3, the loop that links domains D3 and D4, contains a tripeptide which acts as an intracellular plug that closes the pore after activation, thus inducing the channel to enter the inactive state. After inactivation, these channels further undergo conformational change to restore their resting state and become available for activation. This period is referred to as recovery from inactivation (repriming). Different channels reprime at different rates, and repriming in SNS is relatively rapid.

Based on amino acid similarities, the voltage-gated sodium channel family has been further subdivided into two subfamilies (Felipe et al. (1994) J. Biol. Chem. 269, 30125-30131). Eight of the nine cloned channels belong to subfamily 1. They share many structural features, particularly in their S4 transmembrane segments. However, some of them have been shown to have distinct kinetic properties of inactivation and repriming. Only a single channel of subfamily 2, also referred to as atypical channels, has been identified in human, rat and murine tissues. This subfamily is primarily characterized by reduced numbers of basic residues in its S4 segments, and thus is predicted to have different voltage-dependence compared to subfamily 1. The physiological function of subfamily 2 channels is currently unknown because its electrophysiological properties have not yet been elucidated.

The blocking of voltage-gated sodium channels by tetrodotoxin, a neurotoxin, has served to functionally classify these channels into sensitive (TTX-S) and resistant (TTX-R) phenotypes. Two mammalian TTX-R channels have so far been identified, one specific to the cardiac muscle and to very limited areas of the central nervous system (CNS) and the second, SNS, is restricted to peripheral neurons (PNS) of the dorsal root ganglia (DRG) and trigeminal ganglia. Specific amino acid residues that confer resistance or sensitivity to TTX have been localized to the ion selectivity filter of the channel pore.

Naᵥ1.9 (formerly known as NaN, for New and Nociceptive) is a voltage-gated sodium channel α-subunit, which is TTX-R and preferentially expressed within small (<30 μm) sensory neurons of dorsal root ganglia (DRG) and trigeminal ganglia, but not in CNS neurons and glia or muscle. Low levels of Naᵥ1.9 are detectable in cerebrum and retina, but Naᵥ1.9 is not detectable in cerebellum, spinal cord, or in satellite or Schwann cells within DRG.

All of the relevant landmark sequences of voltage-gated sodium channels are present in Naᵥ1.9 at the predicted positions, indicating that Naᵥ1.9 belongs to the sodium channel family. But Naᵥ1.9 is distinct from all other previously identified sodium channels, sharing a sequence identity of less than 53% with each one of them. Naᵥ1.9 is distinct from Naᵥ1.8 (formerly SNS), the only other TTX-R sodium channel subunit that had been identified in PNS. In normal expression of Naᵥ1.9, surface expression of Naᵥ1.9 appears to be related to its association with the cell adhesion molecule contactin/F3 (Liu et al. (2001) J. Biol. Chem. 276, 46553-46561), as co-expression of the two proteins increases the surface expression of Naᵥ1.9. This expression can be modulated by neurotrophins, such as nerve growth factor (NGF) and glial cell-derived neurotrophic factor (GDNF) as examples (Dib-Hajj (2002) manuscript under review).

Previously, a Naᵥ1.9 has been cloned without using any primers or probes that are based upon or specific to SNS (WO 97/01577). Moreover, Naᵥ1.9 and Naᵥ1.8 share only 47% similarity of their predicted open reading frame (ORF), comparable to the limited similarity of Naᵥ1.9 to all subfamily 1 members. The low sequence similarity to existing α-subunits clearly identifies Naᵥ1.9 as a novel gene, not simply a variant of an existing channel. Sequence variations compared to the other voltage-gated channels indicate that Naᵥ1.9 may be the prototype of a novel and previously unidentified, third class of TTX-R channels that may possess distinct properties compared to Naᵥ1.8. Naᵥ1.9 and Naᵥ1.8, which are present in nociceptive DRG and trigeminal neurons, may respond to pharmacological interventions in different ways. The preferential expression of Naᵥ1.9 in sensory DRG and trigeminal neurons provides a target for selectively modifying the behavior of these nerve cells while not affecting other nerve cells in the brain and spinal cord. A further elucidation of the properties of Naᵥ1.9 channels will be important to understand more fully the effects of drugs designed to modulate the function of the “TTX-R” currents which are characteristic of DRG nociceptive neurons and which contribute to the transmission of pain messages, and to abnormal firing patterns after nerve injury and in other painful conditions.

### SUMMARY OF THE INVENTION

The present invention concerns functional expression of an isolated nucleic acid sequence which encodes a voltage gated sodium channel that is preferentially expressed in dorsal root ganglia or trigeminal ganglia (the Naᵥ1.9 channel) and recombinant cells which are transiently or stably transfected with and express Naᵥ1.9.

In some embodiments, the invention encompasses an expression vector comprising a nucleic acid sequence which encodes a mammalian Naᵥ1.9 sodium channel protein or a fragment thereof, wherein the expression vector produces a sodium current when transfected in a cell. In a preferred embodiment, the mammalian Naᵥ1.9 is selected from the group consisting of human, rat or murine Naᵥ1.9. In one embodiment, rat Naᵥ1.9 comprises an amino acid sequence selected from the group consisting of amino acid residues 1 to 1765 of SEQ ID NO: 2, an amino acid sequence comprising a fragment of amino acid residues 1 to 1765 of SEQ ID NO: 2, and amino acid residues 1 to 1765 of SEQ ID NO: 2 comprising at least one conservative substitution.

In another embodiment of the invention, the human Naᵥ1.9 comprises an amino acid sequence selected from the group consisting of amino acid residues 1 to 1791 of SEQ ID NO: 4, an amino acid sequence comprising a fragment of amino acid residues 1 to 1791 of SEQ ID NO: 4, and amino acid residues 1 to 1791 of SEQ ID NO: 4 comprising at least one conservative substitution.

In yet another embodiment, the murine Naᵥ1.9 comprises an amino acid sequence selected from the group consisting of amino acid residues 1-1765 of SEQ ID NO: 6, an amino acid sequence comprising a fragment of amino acid residues 1-1765 of SEQ ID NO: 6, and amino acid residues 1-1765 of SEQ ID NO: 6 comprising at least one conservative substitution.

In some embodiments, the expression vector is an expression plasmid. In a preferred embodiment, the expression plasmid is a low copy number expression plasmid. In a more preferred embodiment, the expression plasmid comprises a promoter sequence operably linked to the Naᵥ1.9 sequence. In one embodiment, the promoter sequence is a CMV promoter. In a even more preferred embodiment, the expression plasmid further comprising a selectable marker under the control of a second promoter sequence. In one embodiment, the selectable marker is a neomycin resistance gene.

The invention includes expression plasmids containing rat Naᵥ1.9. In a preferred embodiment, the rat Naᵥ1.9 comprises SEQ ID NO: 20. In yet another preferred embodiment, the expression plasmid further comprises at the 5′ end adjacent to said open reading frame encoding said SEQ ID NO: 20 at least about 1-100 of the untranslated nucleic acid residues which are 5′ to the open reading frame of SEQ ID NO: 19. In some embodiments the expression plasmid, comprises at least about 1-50 of said 5′ untranslated nucleic acid residues, while in a preferred embodiment the expression plasmid comprises 38 of said 5′ untranslated nucleic acid residues. In one embodiment, the expression plasmid is prNaN.

In some embodiments, the expression plasmids of the invention further comprise a nucleic acid sequence which encodes a histidine tag sequence, wherein the expression of said tag is under the control of the same promoter which controls expression of said open reading frame. In yet other embodiments, the invention includes expression plasmids further comprising a nucleic acid sequence which encodes a green fluorescent protein (GFP) label polypeptide, wherein the expression of said GFP is under the control of the same promoter which controls expression of said open reading frame. In a preferred embodiment, the expression plasmid is pCMV-rNaN-GFP. The invention further includes a recombinant cell comprising the plasmids of the invention. In a preferred embodiment, the recombinant cell comprises the pCMV-rNaN-GFP plasmid.

The invention includes an expression plasmid comprising a nucleic acid sequence which encodes a mammalian Naᵥ1.9 sodium channel protein or a fragment thereof, wherein said mammalian Naᵥ1.9 sodium channel protein or fragment thereof does not produce a sodium current in a transfected cell. In some embodiments, the expression plasmid is a rat Naᵥ1.9 which comprises an open reading frame encoding SEQ ID NO: 2. In another embodiment, SEQ ID NO: 2 comprises at least one mutation selected from the group consisting of a serine to proline change at amino acid position 962, a leucine to proline change at amino acid position 1282 and a deletion of amino acid residues 1000-1010. In a preferred embodiment, the expression plasmid is pLG338XM-rNaN. The invention also includes a recombinant cell comprising any of the aforementioned plasmids including pLG338XM-rNaN.

The invention encompasses a method of making a cell or cell line that produces a Naᵥ1.9 sodium channel-dependent sodium current comprising providing a cell which has been transfected with an expression vector which comprises a nucleic acid sequence which encodes the Naᵥ1.9 sodium channel protein, and culturing said cell under conditions which allow expression of the Naᵥ1.9 sodium channel protein to produce a sodium current in the transfected cell. In some embodiments, the mammalian Naᵥ1.9 is selected from the group consisting of human, rat and murine Naᵥ1.9. In another embodiment, the expression vector in the above method is an expression plasmid. In a preferred embodiment, the expression plasmid is selected from the group consisting of prNaN, a plasmid comprising SEQ ID NO: 19, and pCMV-rNaN-GFP.

The invention includes a method of screening for an agent that modulates sodium current in a cell comprising exposing a the cell or cell line produced by the aforementioned method to the agent; and measuring sodium current following exposure to the agent, wherein an alteration in the level of sodium current is indicative of an agent capable modulating sodium current in a cell.

The invention also includes a recombinant cell comprising any of the expression vectors of the invention. In some embodiments, the cell transiently expresses the mammalian Naᵥ1.9 sodium channel protein or fragment thereof which produces a sodium current in a transfected cell. In other embodiments, the cell stably expresses the mammalian Naᵥ1.9 sodium channel protein or fragment thereof which produces a sodium current in a transfected cell. In one embodiment, the expression vector is a viral vector selected from the group consisting of adenovirus, adeno-associated virus, and baculovirus.

### BRIEF DESCRIPTION OF THE DRAWINGS

FIG. 1 shows the non-expression plasmid pLG338-rNaN comprising rNaN inserted into a modified pLG338 vector.

FIG. 2 shows the low copy number expression plasmid pLG338XM.

FIG. 3 shows the expression plasmid pLG338XM-rNaN, constructed by cloning the rNaN ORF from pLG338-rNaN into pLG338XM to create the mammalian expression construct.

FIG. 4 shows the analysis of rNaN expression in HEK293 cells using RT-PCR and Western blot analysis. (A) RT-PCR analysis. The expected product of rNaN amplification (318 bp) is detected in the (+)RT template from rat DRG and transfected HEK293 cells (lanes 1 and 4, respectively). No amplification signal is detectable in (+)RT untransfected HEK293 cells (lane 2), (−)RT transfected HEK293 cells (lane 3) or control template (lane 5). “M” lane is a 1 kilobase ladder. (B) Western blot analysis. Membrane fraction from untransfected HEK293 cells (lane 1), transfected HEK293 cells (lane 2) and rat DRG tissue (lane 3) were analyzed for the presence of Naᵥ1.9 signal. A protein species of about 210 kDa is observed in native DRG tissue and transfected HEK293 cells but is missing from control HEK 293 cells. The size of the protein is in agreement with the predicted molecular weight of 201 kDa.

FIG. 5 shows the GFP-NaN construct expressed in DRG neurons in vitro. GFP (A) and Naᵥ1.9 (B) are co-expressed in small DRG neurons. Note that DRG neurons not transfected with GFP do not exhibit NaN/SNS2/Naᵥ1.9 immunoreactivity (arrows). (C) Nomarski image of field shown in (A) and (B). Scale bar, 25 μm.

FIG. 6 shows the promotorless rNaN2 clone pLG338-rNaN-BGHneo with BGHneo.

FIG. 7 shows the promotorless rNaN-6×His-GFP fusion clone.

FIG. 8 shows the expression vector for rNaN-6×His-GFP fusion construct.

FIG. 9 shows current signatures of (A) HEK293 cells and (B) mouse SNS-null DRG neurons which were transfected with the pCMV-rNaN-GFP construct and characterized for the production of currents. Current signatures characteristic of native currents in DRG cells (C) were detected and measured.

FIG. 10 shows the expression vector prNaN which expresses the full correct sequence of rNaN (SEQ ID NO: 20) at the amino acid level.

FIG. 11 shows the expression of RNA, protein and sodium current in stable HEK293-derived cell lines transfected with rNaᵥ1.9. (A) Full length rNaᵥ1.9 cDNA was amplified by RT-PCR from templates of the S2 and S6 cell lines. (B) Immunoblot assay to detect Naᵥ1.9 protein in the membrane fractions of stable cell lines S1, S2, S5 and S6. (C) Representative single cell current signature of transfected HEK293 cell lines stably expressing Naᵥ1.9 sodium channels as recorded in a whole-cell patch-clamp.

FIG. 12 shows the expression of Naᵥ1.9 channel in HEK 293 cell transfected with human NaN/Naᵥ1.9. (A) Full length (lane 1) or overlapping segments of human NaN/Naᵥ1.9 cDNA (lanes 2-4) were amplified by RT-PCR using primers pairs (a-d) described in Table 2. Lane 1 shows a single amplification product from a reaction of primer pair (a), consistent with an amplification product of 5400 bases. Lane M contains 1 Kbp molecular weight markers. Lanes 2-4 show the amplification products of primer pairs (b-d), respectively, which are consistent with the predict amplicon lengths of 1936, 1970 and 1539 bases, respectively.

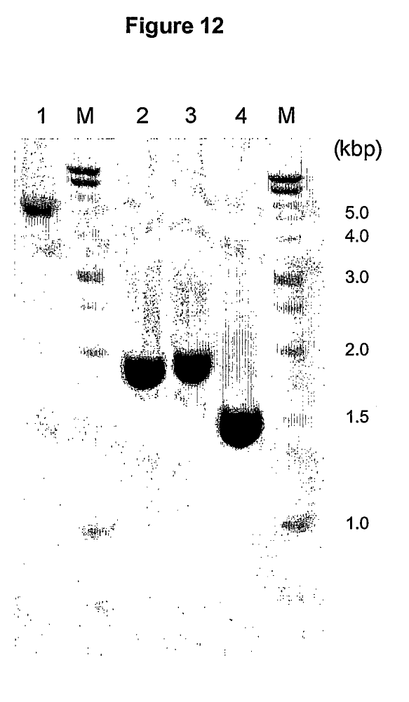

FIG. 13 shows the expression of Naᵥ1.9 channel in HEK 293 cell transfected with murine Naᵥ1.9. Overlapping segments of which spans the entire length of the mouse NaN/Naᵥ1.9 cDNA were amplified by RT-PCR using primers pairs (e-f) described in Table 2. Lane M contains 1 Kbp molecular weight markers. Lanes 1-3, show the amplification products of primer pairs “e-f”, respectively, which consistent with the predict amplicon lengths of 1813 bp, 2034 bp, and 1584 bp, respectively.

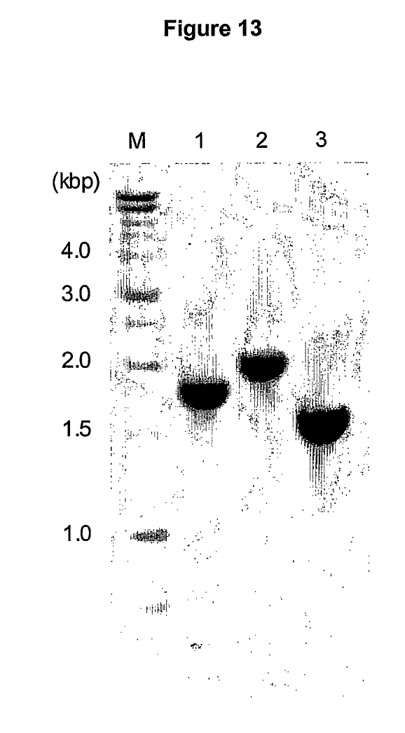

FIG. 14 shows sodium channel human and murine Naᵥ1.9 immunostaining in HEK cells. (A) HEK cells were transfected with human Naᵥ1.9 construct and reacted with anti-human Naᵥ1.9 antibody. Transfected cells exhibit intense Naᵥ1.9 immunofluorescence, while adjacent non-transfected cells do not display Naᵥ1.9 immunolabeling. (B) HEK cells were transfected with mouse Naᵥ1.9 construct and reacted with anti-mouse Naᵥ1.9 antibody. Transfected cells exhibit intense Naᵥ1.9 immunofluorescence, while adjacent non-transfected cells do not display Naᵥ1.9 immunolabeling. (A)′ and (B)′ are Nomarski bright field images of fields in (A) and (B), respectively, showing the presence of many un-transfected HEK 293 cells.

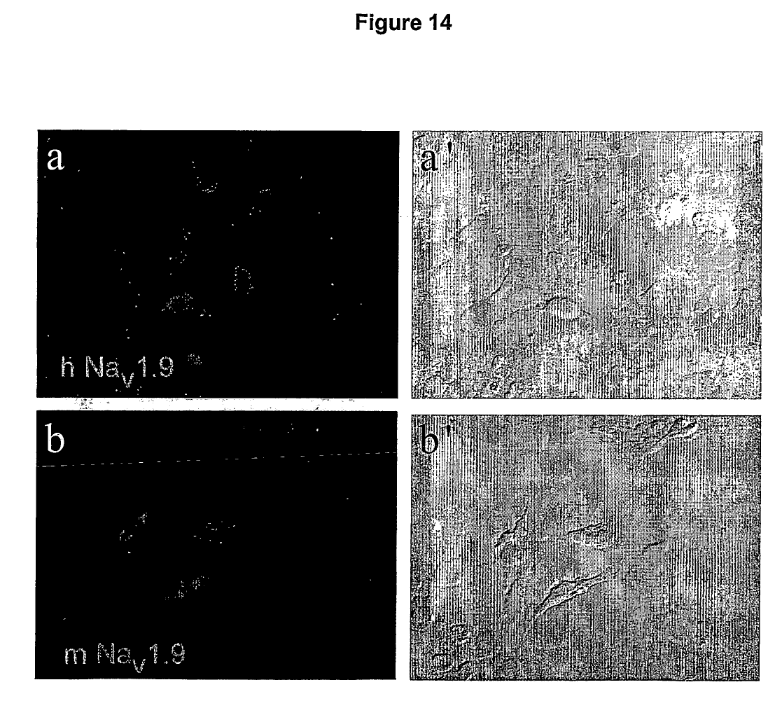

### DETAILED DESCRIPTION OF THE INVENTION

The present invention relates to expression vectors comprising nucleic acid sequences encoding the Naᵥ1.9 sodium channel α-subunit, which is TTX-R, voltage-gated, and preferentially expressed in sensory neurons innervating the body (dorsal root ganglia or DRG) and the face (trigeminal ganglia) and cell lines transiently or stably expressing Naᵥ1.9.

Definitions

As used herein, the term “fusion protein” refers to a C-terminal to N-terminal fusion of one protein molecule to another protein molecule. The fusion proteins of the present invention typically include constructs in which a linker peptide sequence is utilized. The protein molecules are fused to one another in such a manner as to produce a single fusion protein comprised of two or more protein molecules.

As used herein, the term “modulate” refers to up- or down-regulation of the expression and/or activity of a particular receptor, its ligand or current flow. For example an agent might modulate sodium current flow by inhibiting (decreasing) or enhancing (increasing) sodium current flow. Similarly, an agent might modulate the level of expression of the Naᵥ1.9 sodium channel or the activity of the Naᵥ1.9 channels that are expressed.

As used herein, the term “sodium current” or “Na⁺ current” refers to the flow of sodium ions across a cell membrane, often through channels (specialized protein molecules) that are specifically permeable to certain ions, in this case sodium ions.

As used herein, the term “voltage gated” refers to an ion channel that opens when the cell reaches a certain membrane potential. Voltage-sensitive sodium channels open when the membrane is depolarized. They then permit sodium ions to flow into the cell, producing further depolarization. This permits the cell to an action potential.

As used herein, the terms “TTX-R” and “TTX-S” refer to current flow through a cell membrane that is resistant or sensitive to the presence of tetrodotoxin, respectively (a neurotoxin produced in certain species).

As used herein, the term “modulator of sodium current flow” generally refers to when an agent has altered such current flow relative to a control cell not exposed to that agent. A preferred modulator will selectively modulate such current flow, without affecting the current flow of other sodium channels; or it will modulate sodium current in the channel of interest to a much larger extent than in other channels.

As used herein, the term “inhibitor of sodium current flow” generally refers to when an agent has decreased such current flow relative to a control cell not exposed to that agent. A preferred inhibitor will selectively inhibit such current flow, without affecting the current flow of other sodium channels; or it will inhibit sodium current in the channel of interest to a much larger extent than in other channels.

As used herein, the term “enhancer of sodium current flow” generally refers to when an agent has increased such current flow relative to a control cell not exposed to that agent. A preferred agent will selectively increase such current flow, without affecting the current flow of other sodium channels; or it will increase sodium current in the channel of interest to a much larger extent than in other channels.

As used herein, the term “specifically hybridizes” refers to nucleic acids which hybridize under stringent, preferably moderately stringent, or more preferably highly stringent, conditions to the nucleic acids encoding the Naᵥ1.9 sodium channel, such as the DNA sequence of SEQ ID NOs: 1, 3, 5 or 19. Examples of stringent conditions are described in Sambrook et al. (1989) Molecular Cloning—A Laboratory Manual, Cold Spring Harbor Laboratory Press. The term “moderately stringent conditions” means the identification of nucleic acid sequences having at least about 70% identity to the Naᵥ1.9 sequences of the invention and that nucleic acid molecule hybridization is carried in a buffer consisting of 0.1% SDS, 200 mM NaCl, 6 mM Na₂HPO₄, 2 mM EDTA (pH 6.8). The term “highly stringent conditions” means the identification of nucleic acid sequences having at least about 90% identity to the Naᵥ1.9 sequences of the invention and that hybridization is carried in a buffer consisting of 0.1% SDS, 10 mM NaCl, 0.3 mM Na₂HPO₄, 0.1 mM EDTA (pH 6.8).

As used herein, the term “preferentially expressed” refers to when a voltage gated sodium channel is expressed in the defined tissues in detectably greater quantities than in other tissues. For instance, a voltage gated sodium channel that is preferentially expressed in dorsal root ganglia or trigeminal ganglia is found in detectably greater quantities in dorsal root ganglia or trigeminal ganglia when compared to other tissues or cell types. The quantity of the voltage gated sodium channel may be detected by any available means, including the detection of specific RNA levels and detection of the channel protein with specific antibodies.

As used herein, the term “Naᵥ1.9 protein” refers to a sodium channel protein preferentially expressed in dorsal root ganglia and capable of producing a tetrodotoxin resistant sodium current. Examples include, but are not limited to, human (SEQ ID NO: 4), murine (SEQ ID NO: 6) and rat (SEQ ID NO: 2) Naᵥ1.9 proteins. As used herein, the term “Naᵥ1.9” is synonymous with the term “NaN” as is well known in the field of the invention.

As used herein, the term “vector” refers to any genetic element, e.g., a plasmid, a chromosome, a virus, behaving either as an autonomous unit of polynucleotide replication within a cell. Suitable vectors include, but are not limited to, plasmids, bacteriophages, cosmids and retroviruses. Vectors will contain polynucleotide sequences which are necessary to effect ligation or insertion of the vector into a desired host cell. Such sequences differ depending on the host organism and will include promoter sequences to effect transcription, enhancer sequences to increase transcription, ribosomal binding site sequences, and transcription and translation termination sequences.

As used herein, the term “Naᵥ1.9 vector” refers to a vector comprising a nucleic acid sequence encoding any Naᵥ1.9 protein including, but not limited to, human (SEQ ID NO: 4), murine (SEQ ID NO: 6) and rat (SEQ ID NO: 2). Naᵥ1.9 vectors of the invention are modified to effectuate expression of a Naᵥ1.9 protein capable of producing a sodium current when expressed in a cell. Examples of such modifications include, but are not limited to, modifications in the nucleic acid sequence encoding the Naᵥ1.9 protein, including non-sense or silent mutations, incorporation of non-translated upstream sequences capable of effecting the expression of Naᵥ1.9 or the copy number of the Naᵥ1.9 vector, and/or incorporation of any nucleotide sequence into the Naᵥ1.9 vector which effects Naᵥ1.9 expression or Naᵥ1.9 vector copy number.

As used herein, the term “low copy number recombinant expression vector” refers to an expression vector which exists in fewer than about 10 to 20 copies per cell, preferably about five copies per cell, more preferably about two to three copies, or less, per cell.

As used herein, the term “host cell” generally refers to prokaryotic or eukaryotic organisms or cells and includes any transformable or transfectable organism or cell which is capable of expressing a protein and can be, or has been, used as a recipient for expression vectors or other transferred DNA. Host cells can be used for the production of protein for purification of Naᵥ1.9 or for membrane expression of Naᵥ1.9 in order to use transformed cells for measurement of membrane potentials. Preferred eukaryotic host cells include, but are not limited to, yeast, Chinese hamster ovary (CHO) cells, established neuronal cell lines, pheochromocytomas, neuroblastomas fibroblasts, HaLa cells, and rhabdomyosarcomas. Most preferred as eukaryotic host cells are HEK293 cells and dorsal root ganglion cells.

As used herein, the term “transformed” refers to any known method for the insertion of foreign DNA or RNA sequences into a prokaryotic host cell. The term “transfected” refers to any known method for the insertion of foreign DNA or RNA sequences into a eukaryotic host cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include but is not limited to, transforming bacteria with a bacteriophage vector, plasmid vector, or cosmid DNA; transfecting yeast with yeast vectors; infecting insect cell lines with virus (e.g., baculovirus); and transfecting mammalian cell lines with plasmid or viral expression vectors, or with a recombinant virus. Examples of suitable viral vectors for transfection of mammalian cells include, but are not limited to, adenovirus, adeno-associated virus, polio virus, SV40, vaccinia, retrovirus, HIV, and BIV or baculovirus pseudotyped with an envelope protein (such as the vesicular stomatitis virus G protein, VSVG). Transfection with a viral vector may also include the use of a helper virus, a vector able to trans-complement in full or in part a recombinant viral vector defective for replication. Suitable plasmid vectors may include, but are not limited to, pBR322, pAC105, pVA51, pACYC177, pKH47, pACYC184, pUB110, pMB9, pBR325, Co1 E1, pSC101, pBR313, pML21, RSF2124, pCR1, RP4, pBR328 and the like. Transfection can also be in the form of direct uptake of the polynucleotide, such as, for example, lipofection or microinjection. Transfection can also be in the form of biolistic transfection, wherein the vector is coated onto gold particles and delivered into the host cell using a gene gun.

Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake. It is to be understood that this invention is intended to include other forms of expression vectors, host cells, and transformation techniques which serve equivalent functions and which become known to the art hereto.

As used herein, the term “recombinant” refers to a protein that is derived from recombinant (e.g., microbial or mammalian) expression systems. “Microbia” refers to recombinant proteins made in bacterial, fungal (e.g., yeast) or insect expression systems. Proteins expressed in most bacterial cultures will be free of glycan. Protein expressed in yeast may have a glycosylation pattern different from protein expressed in mammalian cells.

As used herein, the term “recombinant expression vector” refers to a replicable DNA construct used either to amplify or to express DNA which encodes a protein. The recombinant expression vector includes a transcriptional unit comprising an assembly of (1) a genetic element or elements having a regulatory role in gene expression, for example, promoters or enhancers; (2) a structural or coding sequence which is transcribed into mRNA and translated into protein; and (3) appropriate transcription and translation initiation and termination sequences. Structural elements intended for use in yeast expression systems preferably include a leader sequence enabling extracellular secretion of translated protein by a host cell. Alternatively, where recombinant protein is expressed without a leader sequence or transport sequence, it may include an N-terminal methionine residue. This residue may optionally be subsequently cleaved from the expressed recombinant protein to provide a final product.

Recombinant DNA Molecules

The present invention further provides recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see Sambrook et al. (1989) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad), pPL and pKK223 available from (Pharmacia).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies Inc.), pTDT1 (ATCC), the vector pCDM8, Rc/CMV (Invitrogen), the plasmid pLG338 described herein, and the like eukaryotic expression vectors. Examples of suitable viral vectors for transfection of mammalian cells include, but are not limited to, adenovirus, adeno-associated virus, polio virus, SV40, vaccinia, retrovirus, HIV, and BIV or baculovirus pseudotyped with an envelope protein (such as the vesicular stomatitis virus G protein, VSVG). Transfection with a viral vector may also include the use of a helper virus, a vector able to trans-complement in full or in part a recombinant viral vector defective for replication. In some cases, transfection comprises the use of two or more vectors, wherein overlapping Naᵥ1.9 segments of the coding sequence are contained on the separate vectors. The separate vectors are transfected into the host cell and the segments are recombined within the host cell to generate a full length coding sequence in the host cell which expresses Naᵥ1.9.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene (Southern et al. (1982) J. Mol. Anal. Genet. 1, 327-341). Alternatively, the selectable marker can be present on a separate plasmid, and the two vectors are introduced by co-transfection of the host cell, and selected by culturing in the appropriate drug for the selectable marker.

Recombinant Fusion Proteins

A DNA sequence encoding a fusion protein is constructed using recombinant DNA techniques to assemble separate DNA fragments encoding proteins into an appropriate expression vector. For example, the 3′ end of a DNA molecule encoding a protein is ligated to the 5′ end of a second DNA molecule encoding the same or a substantially similar protein, with the reading frames of the sequences in phase to permit mRNA translation of the sequences into a single biologically active fusion protein. The DNA molecules are joined in tandem, meaning that the DNA molecules are joined in succession, one after the other. The regulatory elements responsible for transcription of DNA into mRNA are retained on the first of the two DNA sequences while binding signals or stop codons, which would prevent read-through to the second DNA sequence, are eliminated. Conversely, regulatory elements are removed from the second DNA sequence while stop codons required to end translation are retained.

As described herein, means are provided for linking protein molecules, preferably via a linker sequence. The linker sequence separates the protein molecules by a distance sufficient to ensure that each protein molecule properly folds into its secondary and tertiary structures. Suitable linker sequences (1) adopt a conformation suitable to result in a fusion protein with increased biological activity, (2) do not exhibit a propensity for developing an ordered secondary structure which could impair the biological functions of the protein molecules, and (3) have minimal hydrophobic or charged character which could impair the biological functions of Naᵥ1.9 proteins. For example, a suitable linker will produce a fusion protein where interaction of the protein components results in increased biological activity. The linker conformation can be flexible or rigid, depending on the final conformation of the fusion required to result in increased biological activity. An example of a more rigid linker would be a linker with an alpha-helix that would not allow free rotation of the linked protein components. Typical surface amino acids in flexible protein regions include glycine, asparagine and serine. Virtually any permutation of amino acid sequences containing glycine, asparagine and serine would be expected to satisfy the above criteria for linker sequence. Other near neutral amino acids, such as threonine and alanine, may also be used in the linker sequence.

The length of the linker sequence may vary without significantly affecting the biological activity of the fusion protein. Generally, the Naᵥ1.9 protein will be separated by a linker sequence having a length of about 6 amino acids to about 20 amino acids, although longer linker sequences may be used, for example, a full-length polypeptide can comprise the linker. Preferably, the linker sequence is about 8 amino acids in length. In the most preferred aspects of the present invention, the linker sequence is an Ile-Asp-polyHis sequence (SEQ ID NO: 21). The linker sequence is incorporated into the fusion protein using any method known in the art.

Also encompassed by the present invention are fusion proteins comprising biologically active fragments, analogs, mutants, variants or derivatives of the naturally occurring proteins described herein. Biologically active fragments, derivatives, analogs, variants and mutants of the naturally occurring proteins are also referred to herein as substantially similar proteins of the naturally occurring protein. However, the level of biological activity of fragments, analogs, mutants, variants or derivatives of the naturally-occurring protein need not be identical to the activity of the naturally-occurring protein (also referred to herein as the parent protein). For example, a fragment of a Naᵥ1.9 protein may exhibit only 50-80% of the activity of the naturally occurring Naᵥ1.9 protein. Tests to determine biological activity are well known to those of skill in the art and can include, for example, measuring the extent of sodium current flow, membrane potential and receptor binding.

The present invention also provides fusion proteins with or without associated native-protein glycosylation. Expression of DNA encoding the fusion proteins in bacteria such as E. coli provides non-glycosylated molecules. Functional mutant analogs having inactivated N-glycosylation sites can be produced by oligonucleotide synthesis and ligation or by site-specific mutagenesis techniques. These analog proteins can be produced in a homogeneous, reduced carbohydrate form in good yield using yeast expression systems. N-glycosylation sites in eukaryotic proteins are characterized by the amino acid triplet Asn-A₁-Z, where A₁ is any amino acid except proline and Z is serine or threonine. In this sequence, asparagine provides a side chain amino group for covalent attachment of carbohydrate. Such a site can be eliminated by substituting another amino acid for asparagine (Asn) or for residue Z, deleting asparagine (Asn) or Z, or inserting a non-Z amino acid between A₁ and Z, or an amino acid other than asparagine between asparagine and A₁.

Derivatives and analogs can be obtained by mutations of the fusion protein. A derivative or analog, as referred to herein, is a polypeptide comprising an amino acid sequence that shares sequence identity, or similarity, with the full-length sequence of the wild type (or naturally occurring protein), except that the derivative or analog has one or more amino acid sequence differences attributable to a deletion, insertion and/or substitution.

Bioequivalent analogs of proteins can be constructed by, for example, making various substitutions of residues or sequences. For example, cysteine residues can be deleted or replaced with other amino acids to prevent formation of incorrect intramolecular disulfide bridges upon renaturation. Other approaches to mutagenesis involve modification of adjacent dibasic amino acid residues to enhance expression in yeast systems in which KEX2 protease activity is present. Generally, substitutions should be made conservatively; i.e., the most preferred substitute amino acids are those having physicochemical characteristics resembling those of the residue to be replaced. Similarly, when a deletion or insertion strategy is adopted, the potential effect of the deletion or insertion on biological activity should be considered. Due to degeneracy of the genetic code, there can be considerable variation in nucleotide sequences encoding the same amino acid sequence.

Mutations in nucleotide sequences constructed for expression of analogs must, of course, preserve the reading frame phase of the coding sequences and preferably will not create complementary regions that could hybridize to produce secondary mRNA structures such as loops or hairpins which would adversely affect translation of the mRNA. Alternatively, mutations could introduce secondary structure that would result in higher translational efficiency. Although a mutation site may be predetermined, it is not necessary that the nature of the mutation per se be predetermined. For example, in order to select for optimum characteristics of mutants at a given site, random mutagenesis may be conducted at the target codon and the expressed mutants screened for the desired activity.

Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene having particular codons altered according to the substitution, deletion, or insertion required. Exemplary methods of making the alterations set forth above are disclosed by Walder et al. (1986) Gene 42, 133-134 and Bauer et al. (1985) Gene 37, 73-75.

The present invention also provides recombinant expression vectors which include synthetic or cDNA-derived DNA fragments encoding fusion proteins comprising DNA encoding two or more linked proteins operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation, as described in detail below. The ability to replicate in a host, usually conferred by an origin of replication, and a selection gene to facilitate recognition of transformants may additionally be incorporated. Operably linked indicates that components are linked in such a manner that expression of the DNA encoding a fusion protein is controlled by the regulatory elements. Generally, operably linked means contiguous.

Transformed host cells are cells into which fusion protein vectors have been introduced by infectious or non-infectious methods. Transformed host cells ordinarily express the desired fusion protein, but host cells transformed for purposes of cloning or amplifying DNA do not need to express the protein. Suitable host cells for expression of fusion protein include prokaryotes, yeast or higher eukaryotic cells under the control of appropriate promoters. Prokaryotes include gram negative or gram positive organisms, for example E. coli. Higher eukaryotic cells include established cell lines of mammalian origin as described herein. Cell-free translation systems could also be employed to produce fusion protein using RNA derived from the DNA constructs of the present invention.

Prokaryotic expression vectors generally comprise one or more phenotypic selection markers, for example a gene encoding proteins conferring antibiotic resistance or supplying an autotrophic requirement, and an origin of replication recognized by the host to ensure amplification within the host. Suitable prokaryotic hosts for transformation include, but are not limited to, E. coli, Bacillus subtilis, Salmonella typhimurium, and various species within the genera Pseudomonas, Streptomyces and Staphyolococcus, although others may also be employed as a matter of choice.

Useful expression vectors for bacterial use can comprise a selectable marker and bacterial origin of replication derived from commercially available plasmids comprising genetic elements of the well-known cloning vector pBR322 (ATCC). Such commercial vectors include, but are not limited to, pKK223-3 (Pharmacia) and pGEM1 (Promega). These pBR322 sections are combined with an appropriate promoter and the structural sequence to be expressed. pBR322 contains genes for ampicillin and tetracycline resistance and thus provides simple means for identifying transformed cells.

Promoters commonly used in recombinant microbial expression vectors include, but are not limited to, the blactamase (penicillinase) and lactose promoter system (Chang et al. (1978) Nature 275, 615-616 and Goeddel et al. (1979) Nature 281, 544-545), the tryptophan (trp) promoter system (Goeddel et al. (1980) Nuc. Acids Res. 8, 4057-4058) and tac promoter (Sambrook et al. (1989) Molecular Cloning: A Laboratory Manual, Cold Spring Harbor Laboratory Press).

Recombinant fusion proteins may also be expressed in yeast hosts, preferably from the Saccharomyces species, such as S. cerevisiae. Yeast of other genera such as, but not limited to, Pichia or Kluyveromyces may also be employed. Yeast vectors will generally contain an origin of replication from a yeast plasmid or an autonomously replicating sequence (ARS), promoter, DNA encoding the fusion protein, sequences for polyadenylation and transcription termination and a selection gene. Preferably, yeast vectors will include an origin of replication and selectable marker permitting transformation of both yeast and E. coli, e.g., the ampicillin resistance gene of E. coli and S. cerevisiae trp1 gene, which provides a selection marker for a mutant strain of yeast lacking the ability to grow in tryptophan, and a promoter derived from a highly expressed yeast gene to induce transcription of a structural sequence downstream. The presence of the trp1 lesion in the yeast host cell genome then provides an effective environment for detecting transformation by growth in the absence of tryptophan.

Suitable promoter sequences in yeast vectors include the promoters for metallothionein, 3-phosphoglycerate kinase (Hitzeman et al. (1980) J. Biol. Chem. 255, 2073-2075) or other glycolytic enzymes (Hess et al. (1968) J. Adv. Enzyme Reg. 7, 149-150 and Holland et al. (1978) Biochem. 17, 4900-4901), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triose-phosphate isomerase, phosphoglucose isomerase and glucokinase. Preferred yeast vectors can be assembled using DNA sequences from pBR322 for selection and replication in E. coli (Amp gene and origin of replication) and yeast DNA sequences including a glucose-repressible ADH2 promoter and alpha-factor leader, which directs secretion of heterologous proteins, can be inserted between the promoter and the structural gene to be expressed (Kurjan et al. (1982) Cell 30, 933-934 and Bitter et al. (1984) Proc. Natl. Acad. Sci. USA 81, 5330-5331). The leader sequence may be modified to contain, near its 3′ end, one or more useful restriction sites to facilitate fusion of the leader sequence to foreign genes.

Suitable yeast transformation protocols are known to those of skill in the art; an exemplary technique is described by Hinnen et al. (1978) Proc. Natl. Acad. Sci. USA 75, 1929-1930, selecting for Trp⁺ transformants in a selective medium consisting of 0.67% yeast nitrogen base, 0.5% casamino acids, 2% glucose, 10 μg/ml adenine and 20 μg/ml uracil. Host strains transformed by vectors comprising the ADH2 promoter may be grown for expression in a rich medium consisting of 1% yeast extract, 2% peptone and 1% glucose supplemented with 80 μg/ml adenine and 80 μg/ml uracil. Derepression of the ADH2 promoter occurs upon exhaustion of medium glucose. Crude yeast supernatants are harvested by filtration and held at 4° C. prior to further purification. Various mammalian or insect cell culture systems can be employed to express recombinant protein. Baculovirus systems for production of heterologous proteins in insect cells are reviewed by Luckow and Summers (1988) Biotechnology 6, 47-48.

Examples of suitable mammalian host cell lines include, but are not limited to, the COS-7 lines of monkey kidney cells, described by Gluzman (1981) Cell 23, 175-177) and other cell lines capable of expressing an appropriate vector including, for example, HEK cell lines, L cells, C127, 3T3, Chinese Hamster Ovary (CHO), HeLa and BHK cell lines. Mammalian expression vectors may comprise non-transcribed elements such as an origin of replication, a suitable promoter and enhancer linked to the gene to be expressed, and other 5′ or 3′ flanking nontranscribed sequences, and 5′ to 3′ nontranslated sequences, such as necessary ribosome binding sites, a polyadenylation site, splice donor and acceptor sites, and transcriptional termination sequences.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40) and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment that also contains the SV40 viral origin or replication (Fiers et al. (1978) Nature 273, 113-114). Smaller or larger SV40 fragments may also be used, provided the approximately 250 nucleotide sequence extending from the Hind III site toward the BgII site located in the viral origin or replication is included. Exemplary vectors can be constructed as disclosed by Okayama and Berg (1983) Mol. Cell. Biol. 3, 280-281).

Preferred eukaryotic vectors for expression of mammalian DNA include pIXY321 and pIXY344, both of which are yeast expression vectors derived from pBC102.K22 (ATCC) and contain DNA sequences from pBR322 for selection and replication in E. coli (Apr gene and origin of replication) and yeast. Purified mammalian fusion proteins or analogs are prepared by culturing suitable host/vector systems to express the recombinant translation products of the DNA encoding a Naᵥ1.9 protein, which are then purified from culture media or cell extracts. For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Finally, one or more reverse phase high performance liquid chromatography (RP-HPLC) media, e.g., silica gel having pendant methyl or other aliphatic groups, can be employed to further purify a fusion protein composition. Some or all of the foregoing purification steps, in various combinations, can also be employed to provide a homogenous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant fusion proteins can be disrupted by any convenient method, including freeze/thaw cycling, sonication, mechanical disruption, or use of cell lysing agents.

Fermentation of yeast which express fusion proteins as a secreted protein greatly simplifies purification. Secreted recombinant protein resulting from a large-scale fermentation can be purified by methods analogous to those disclosed by Urdal et al. (1984) J. Chromatog. 296, 171-172. Fusion protein synthesized in recombinant culture is characterized by the presence of non-human cell components, including proteins, in amount and of a character which depend upon the purification steps taken to recover the fusion protein from the culture. These components ordinarily will be of yeast, prokaryotic or non-human higher eukaryotic origin and preferably are present in innocuous contaminant quantities, on the order of less than about five percent by scanning densitometry or chromatography. Further, recombinant cell culture enables the production of the fusion protein free of proteins which may be normally associated with EPO as they are found in nature in their respective species of origin, e.g., in cells, cell exudates or body fluids.

Production of Recombinant Proteins

The present invention further provides methods for producing a protein of the invention using nucleic acid molecules herein described. In general terms, the production of a recombinant form of a protein typically involves the following steps:

A nucleic acid molecule is first obtained that encodes a protein of the invention, such as a nucleic acid molecule comprising, consisting essentially of or consisting of SEQ ID NO: 1, or the open reading frame defined by nucleotides 52-5346 (or 5349) of SEQ ID NO: 1, or encoding SEQ ID NO: 2; a nucleic acid molecule comprising, consisting essentially of or consisting of SEQ ID NO: 3, or the open reading frame defined by nucleotides 31-5403 (or 5406) of SEQ ID NO: 3, or encoding SEQ ID NO: 4; a nucleic acid molecule comprising, consisting essentially of or consisting of SEQ ID NO: 5, or the open reading frame defined by nucleotides 19-5313 (or 5316) of SEQ ID NO: 5, or encoding SEQ ID NO: 6; or a nucleic acid molecule comprising, consisting essentially of or consisting of SEQ ID NO: 19, or the open reading frame defined by nucleotides 1-5295 (or 5298) of SEQ ID NO: 19, or encoding SEQ ID NO: 20.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

Methods to Identify Binding Partners

Another embodiment of the present invention provides methods for use in isolating and identifying binding partners using recombinantly expressed Naᵥ1.9. In general, a Naᵥ1.9 protein is mixed with a potential binding partner or an extract or fraction of a cell under conditions that allow the association of potential binding partners with the protein of the invention. After mixing, peptides, polypeptides, proteins or other molecules that have become associated with the Naᵥ1.9 protein are separated from the mixture. The binding partner that bound to the protein of the invention can then be removed and further analyzed. To identify and isolate a binding partner, the entire Naᵥ1.9 protein, for instance a protein comprising the entire amino acid sequence of SEQ ID NO: 2, 4, 6 or 20, can be used. Alternatively, a fragment of the protein can be used, preferably containing all or a portion of the amino acid sequence of SEQ ID NO: 2, 4, 6 or 20.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue from patients with allergic hypersensitivity. Alternatively, cellular extracts may be prepared from normal tissue or available cell lines, particularly granulocytic cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins.

Alternatively, binding partners may be identified using a Far-Western assay according to the procedures of Takayama et al. (1997) Methods Mol. Biol. 69, 171-184 or Sauder et al. (1996) J. Gen. Virol. 77, 991-996 or identified through the use of epitope tagged proteins or GST fusion proteins. Alternatively, the nucleic acid molecules of the invention can be used in a yeast two-hybrid system. The yeast two-hybrid system has been used to identify other protein partner pairs and can readily be adapted to employ the nucleic acid molecules herein described.

Methods to Identify Agents that Modulate Expression

Another embodiment of the present invention provides methods for identifying agents that modulate the expression of a nucleic acid encoding a recombinant Naᵥ1.9 protein such as a protein having the amino acid sequence of SEQ ID NO: 2, 4, 6 or 20. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, cell lines that contain reporter gene fusions between the open reading frame defined by nucleotides 52-5346 of SEQ ID NO: 1, nucleotides 31-5403 of SEQ ID NO: 3, nucleotides 19-5313 of SEQ ID NO: 5, or nucleotides 1-5295 of SEQ ID NO: 19, and any necessary 5′ and 3′ regulatory elements and any assayable fusion partner may be prepared. Numerous assayable fusion partners are known and readily available including, but not limited to, the firefly luciferase gene and the gene encoding chloramphenicol acetyltransferase (Alam et al. (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of a nucleic acid of the invention.

Additional assay formats may be used to monitor the ability of the agent to modulate the expression of a nucleic acid encoding a Naᵥ1.9 protein, such as the Naᵥ1.9 protein having SEQ ID NO: 00. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (1989) Molecular Cloning—A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes that hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (1989) Molecular Cloning—A Laboratory Manual, Cold Spring Harbor Laboratory Press or Ausubel et al. (1995) Current Protocols in Molecular Biology, Greene Publishing Co.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences encoding the Naᵥ1.9 protein can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding the Naᵥ1.9 protein having the sequence of SEQ ID NO: 2, 4, 6 or 20 are identified.

Hybridization for qualitative and quantitative analysis of mRNA may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

In another assay format, cells or cell lines are first identified which express the Naᵥ1.9 protein physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the Naᵥ1.9 protein fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al. (1989) Molecular Cloning—A Laboratory Manual, Cold Spring Harbor Laboratory Press).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the agent-contacted sample compared to the control will be used to distinguish the effectiveness of the agent.

Methods to Identify Agents that Modulate Activity

The present invention provides methods for identifying agents that modulate at least one activity of a recombinant Naᵥ1.9 protein comprising the sequence of SEQ ID NO: 2, 4, 6 or 20. Such methods or assays may utilize any means of monitoring or detecting the desired activity.

In one embodiment, the invention encompasses a method to identify agents that modulate NaN-mediated current. Several approaches can be used to identify agents that are able to modulate (i.e., block or augment) the Na+ current through the NaN sodium channel. In general, to identify such agents, a model cultured cell line that expresses the NaN sodium channel is utilized, and one or more conventional assays are used to measure Na+ current. Such conventional assays include, for example, patch clamp methods, the ratiometric imaging of [Na+]i, and the use of ²²Na and ⁸⁶Rb as described above.

In one embodiment of the present invention, to evaluate the activity of a candidate compound to modulate Na+ current, an agent is brought into contact with a suitable transformed host cell that expresses NaN. After mixing or appropriate incubation time, the Na+ current is measured to determine if the agent inhibited or enhanced Na+ current flow.

Agents that inhibit or enhance Na+ current are thereby identified. A skilled artisan can readily employ a variety of art-recognized techniques for determining whether a particular agent modulates the Na+ current flow.

Because Na+ is preferentially expressed in pain-signaling cells, one can also design agents that block, inhibit, or enhance Na+ channel function by measuring the response of laboratory animals, treated with these agents, to acute or chronic pain. In one embodiment of this aspect of the invention, laboratory animals such as rats are treated with an agent for instance, an agent that blocks or inhibits (or is thought to block or inhibit) Naᵥ1.9. The response to various painful stimuli are then measured using tests such as the tail-flick test and limb withdrawal reflex, and are compared to untreated controls. These methods are described in Wall et al. (1994) Textbook of Pain, Churchill Livingstone Publishers (see chapter 15). In another embodiment of this aspect of the invention, laboratory animals such as rats are subjected to localized injection of pain-producing inflammatory agents such as formalin (Dubuisson et al. (1977) Pain 4, 161-74), Freunds adjuvant (Iadarola et al. (1988) Pain 35, 313-26) or carageenan, or are subjected to nerve constriction (Bennett et al. (1988) Pain 33, 87-107; Kim et al. (1992) Pain 50, 355-363) or nerve transection (Seltzer et al. (1990) Pain 43, 205-218) which produce persistent pain. The response to various normal and painful stimuli are then measured, for example, by measuring the latency to withdrawal from a warn or hot stimulus Wall et al. (1994) Textbook of Pain, Churchill Livingstone Publishers) so as to compare control animals and animals treated with agents that are thought to modify Naᵥ1.9.

The preferred inhibitors and enhancers of Naᵥ1.9 preferably will be selective for the Naᵥ1.9 sodium channel. They may be totally specific (like tetrodotoxin, TTX, which inhibits sodium channels but does not bind to or directly effect any other channels or receptors), or relatively specific (such as lidocaine which binds to and blocks several types of ion channels, but has a predilection for sodium channels). Total specificity is not required for an inhibitor or enhancer to be efficacious. The ratio of its effect on sodium channels vs. other channels and receptors, may often determine its effect and effects on several channels, in addition to the targeted one, may be efficacious Stys et al. (1992) J. Neurophysiol. 67, 236-40.

It is contemplated that modulating agents of the present invention can be, as examples, peptides, small molecules, naturally occurring and other toxins and vitamin derivatives, as well as carbohydrates. A skilled artisan can readily recognize that there is no limit as to the structural nature of the modulating agents of the present invention. Screening of libraries of molecules may reveal agents that modulate Naᵥ1.9 or current flow through it. Similarly, naturally occurring toxins (such as those produced by certain fish, amphibians and invertebrates) can be screened. Such agents can be routinely identified by exposing a transformed host cell or other cell which expresses a sodium channel to these agents and measuring any resultant changes in sodium ion current.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLE 1

Cloning of rNaᵥ1.9 into a Low Copy Number Non-Expression Plasmid

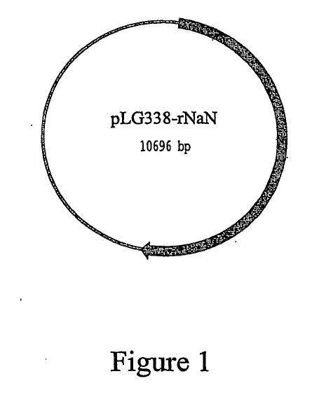

The entire rat Naᵥ1.9 (also referred to as rNaᵥ1.9 or rNaN) was first cloned into a low copy number, non-expression plasmid, pLG338. This was accomplished by first modifying pLG338 to eliminate the unique AflII site by restricting the vector with AflII, polishing the ends with Klenow fragment and religating to produce pLG338ΔAflII. A partial rNaN cDNA clone, L9 (in pTargeT) (Promega), contains a unique 5′ AflII site, 38 of the 5′ untranslated sequence, and nucleotides 1-2964 of the rat NaN/SNS2 open reading frame (ORF). NaN L9 was digested with XhoI (in the polylinker, 5′ to the insert), blunted with Klenow, and then digested with NotI (in the polylinker, 3′ to the insert). The approximately 3 kb 5′ rNaN fragment was gel purified and cloned into pLG338ΔAflII which had been digested with SmaI and NotI (pLG338-5′rNaN). The remaining 3′ rNaN sequence was obtained by PCR amplification using marathon rNaN cDNA as template. The forward primer (5′-gcaagaaatgcaggaggaaaaac-3′; SEQ ID NO: 7) anneals at nucleotide 2565. The reverse primer (5′ataagaat[gcggccgc]caacctg tcacctcgttcagcc-3′; SEQ ID NO: 8) contains a terminal spacer (underlined) and a unique NotI site (brackets), and is predicted to contain 133 bp of the 3′ untranslated nucleotides. The PCR product was digested with BglII and NotI, and the approximately 2.6 kb BglII/NotI fragment was gel purified and cloned into pLG338-5′rNaN, which had been similarly digested and gel purified, to complete the rat Naᵥ1.9 ORF (pLG338-rNaN, FIG. 1).

### EXAMPLE 2

Cloning of rNaᵥ1.9 into a Expression Plasmid

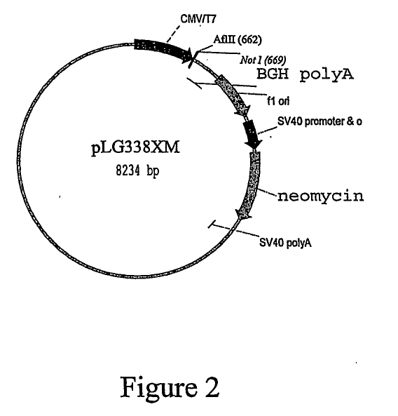

Cloning of neuronal sodium channels into expression vectors is known to be technically difficult, as cloned inserts may become defective through deletion or mutation at the end of the cloning procedure. In order to provide a low copy number vector that expresses rNaN in mammalian cell lines, pLG338ΔAflII was modified by the addition of sequences obtained from the vector pRc/CMV. A fragment that contains the CMV promoter, multiple cloning site, BGH polyA, SV40 promoter, neomycin resistance gene, and SV40 polyA sequences was inserted into pLG338ΔAflII. The presence of the neomycin resistance gene in these constructs provides a means to selectively amplify transfected clones and permits the generation and maintenance of a cell line that stably expresses rNaᵥ1.9. Briefly, pRc/CMV was digested with MluI and SalI enzymes. The 3.1 kb fragment containing all of the above mentioned components was gel isolated and cloned into pLG338ΔAflII which had likewise been digested with MluI and SalI enzymes and gel purified. The multiple cloning site was modified by addition of a linker containing an AflII site 5′ of the NotI site (pLG338XM, FIG. 2).

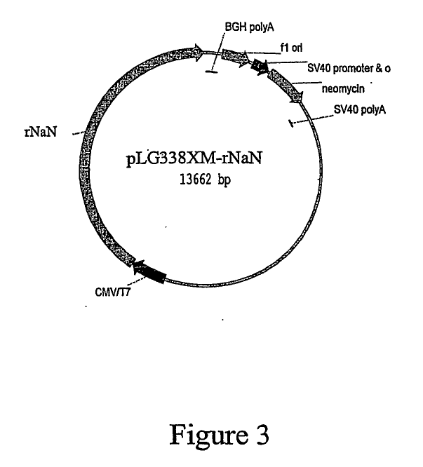

The vectors pLG338XM and pLG338-rNaN were both digested with AflII and NotI enzymes and the rNaN ORF from pLG338-rNaN was cloned into pLG338XM to create the mammalian expression construct pLG338XM-rNaN (FIG. 3).

Sequence analysis of pLG338XM-rNaN revealed deviations from the published rNaᵥ1.9 sequence. Specifically, there is a Ser→Pro change at amino acid 962 in L2, the cytoplasmic loop joining domains 2 and 3 and a Leu→Pro change at amino acid 1282 in DIII-S6. Additionally, an eleven amino acid deletion (1000-1010), also in L2, is detected and is likely due to the utilization of an alternative 3′ splice site of intron 15B. The corresponding regions of the non-expression version pLG338-rNaN were sequenced and contained the identical alterations, thus these may have been present in the initial pool of mRNA templates.

All three of the deviations present in both the expression and non-expression versions of rNaN reside within a region flanked by unique BglII and Bsu36I sites. Additional non-expression clones were sequenced over this region and a clone lacking any changes (at the amino acid level) was identified. When this non-mutated fragment was used to replace the region containing the mutations in pLG338XM-rNaN the construct became unstable. In the non-expression version (pLG338-rNaN) sequence analysis confirmed that this repair was successfully made and is stable after amplification in bacteria (pLG338-rNaN2).

### EXAMPLE 3

Expression of Cloned rNaᵥ1.9 in HEK293 Cells

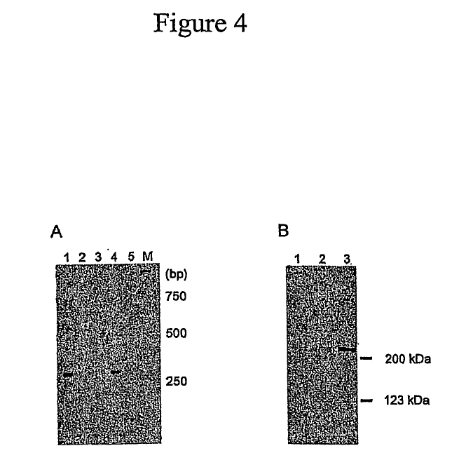

Expression of rNaᵥ1.9 from pLG338XM-rNaN (with the three sequence deviations) in HEK293 cells was confirmed at the RNA and protein levels (FIG. 4). HEK293 cells were transfected with the pLG338XM-rNaN plasmid using the standard calcium-phosphate precipitation method. Control and transfected cells were harvested 18 hours later. Total RNA was isolated using the RNeasy (Qiagen) mini columns according to manufacturer recommendations. The RNA was treated with RNase-free DNase I (Roche) and the RNA was re-purified on RNeasy columns. First strand cDNA was prepared using random hexamer primers as previously described. Forward primer 5′-gaacaaatgtcaagcctttgtgtt-3′ (SEQ ID NO: 9) and reverse primer 5′-cagccatcatgataatcatatttaagac-3′ (SEQ ID NO: 10) amplify an amplicon of 318 nucleotides. RT-PCR shows that a product of the expected size is obtained using rat DRG template and HEK293 transfected with the rNaN construct (FIG. 4A). This product was not detected in reactions using untransfected HEK293 template, a (−) RT transfected HEK293 template or H₂O (FIG. 4A).

Expression of cloned rNaᵥ1.9 at the protein level in transfected HEK293 cells was demonstrated by Western blot analysis. Control and transfected HEK293 cells were harvested eighteen hours post-transfection. The cells were lysed and the membrane fraction was prepared using standard methods. The membrane fraction from rat DRG was processed in a similar manner and served as a positive control. Ten μg of membrane protein of each sample was loaded into wells of a 5% SDS-polyacrylamide gel and subjected to electrophoresis. The proteins were electroblotted onto nitrocellulose membranes. Immunoblotting using rNaᵥ1.9-specific antibody and ECL chemiluminescence detection show the presence of a protein signal of about 210 kDa in both DRG tissue and transfected HEK293 cells, but not in the untransfected HEK293 cells (FIG. 4B).

### EXAMPLE 4

Expression of Cloned rNaᵥ1.9 in Cultured DRG Neurons

Expression of pLG338XM-rNaN was also investigated in cultured mouse DRG neurons following the introduction of this construct using biolistic transformation of these cells. DRG neurons were isolated from SNS-null mice and maintained in culture for three to five days before biolistic transfections. It has been shown that SNS-null DRG neurons express persistent TTX-R sodium currents, but these cells lose transcripts and protein of NaN/SNS2/Na 1.9 after several days in culture. Just before biolistic transfection, the culture medium was removed from the petri dish. The gene gun was held one cm above the cells and a pressure of 120 PSI was used to deliver the gold particles to the cells. A 70-micron nylon mesh (Small Parts, Inc) was placed just in front of the Helios Gene Gun barrel liner in order to achieve a more uniform distribution of gold particles. Within twenty-four hours the cells usually showed expression of green fluorescent protein (GFP), indicating a successful biolistic transfection.

The Helios Gene Gun System (Bio-Rad) was used for biolistic transfection of neurons with DNA coated gold particles. A mixture of 10 μg of pLG338XM-rNaN and 5 μg EGFP (Clontech) DNA in a 50 mM solution of the polyanion spermidine was precipitated onto one micron gold particles using calcium chloride according to the manufacturer recommendations. The DNA gold suspension was washed twice in 100% ethanol and resuspended in 0.05% PVP in ethanol and used for coating the inner wall of a ten inch segment of Tefzel tubing (Bio-Rad Laboratories). The tubing was dried using ultra-pure nitrogen and cut into 0.5 inch cartridges for the Helios gene gun. This process resulted in a density of 1 mg of gold particles per shot and 0.75 μg total DNA per cartridge.

Coverslips with neurons transfected with rNaN construct by gene gun application were processed for immunocytochemistry as follows: (1) complete saline solution, twice, 1 minute each; (2) 4% paraformaldehyde in 0.14 M Sorensen's phosphate buffer, 10 minutes; (3) PBS, three times, 3 minutes each; (4) PBS containing 5% normal goat serum, 2% bovine serum albumin and 0.1% Triton x-100 (blocking solution), 15 minutes; (5) primary antibody (NaN/SNS2/Naᵥ1.9, 1:500, in blocking solution), overnight at 4° C.; (6) PBS, six times, five minutes each; (7) secondary antibody (goat anti-rabbit IgG-Cy3, 1:3000; Amersham); and (8) PBS, six times, five minutes each. A Leitz Aristoplan light microscope equipped with bright field, Nomarski and epifluorescence optics was used for sample observation. The IPLab Spectrum program (Scanalytics) was used for image capture and analysis. Images of fluorescently-labelled neurons were captured with Leica filter blocks N2.1 (Cy3) and L3 (green fluorescent protein).

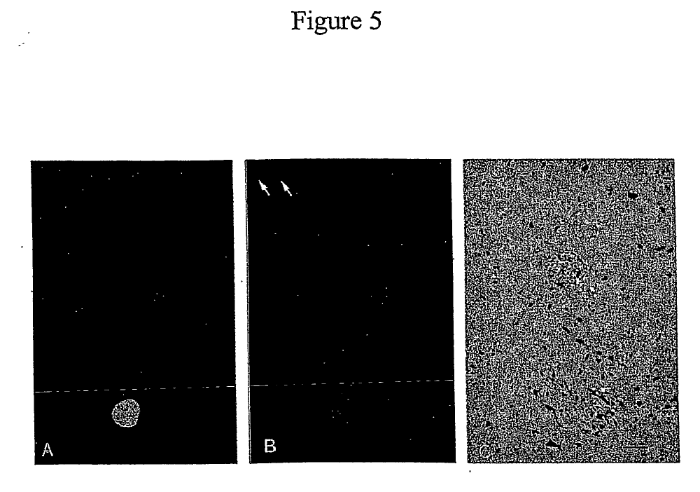

Using the rat NaN/SNS2/Naᵥ1.9-specific antibody, it is demonstrated that this construct produces rat Naᵥ1.9 protein in DRG neurons that have been transformed by pLG338XM-rNaN, while other cells in the same field that did not receive the gold particles do not produce this protein (FIG. 5).

### EXAMPLE 5

Characterization of rNaN Currents in Transfected HEK293 Cells.

HEK293 cells were co-transfected with pLG338XM-rNaN and EGFP (Clontech) using the calcium-phosphate precipitation technique, and green fluorescing cells were selected for electrophysiological analysis forty-eight hours later.

Whole-cell patch-clamp recordings were conducted at room temperature (about 21° C.) using an EPC-9 amplifier, and data was acquired on a Macintosh Quadra 950 computer using the Pulse program (v 7.89, HEKA Electronic, Germany). Fire-polished electrodes (0.8-1.5 MΩ) were fabricated from 1.65-mm Corning 7052 capillary glass using a Sutter P-97 puller (Novato, Calif.). To minimize space clamp problems, only isolated cells with a soma diameter of less than 25 μm were selected for recording. Cells were not considered for analysis if the initial seal resistance was less than 5 GΩ or if they had high leakage currents (holding current >0.1 nA at −80 mV), membrane blebs, or an access resistance greater than 4 MΩ. In previous studies, it was observed that the average access resistance was 2.3:0.6 MΩ(mean±SD, n=116) for cells expressing hNE channels and 2.3±0.6 MΩ (n=52) for cells expressing hSkM1 channels. Voltage errors were minimized using 80% series resistance compensation and the capacitance artifact was canceled using the computer-controlled circuitry of the patch clamp amplifier. Linear leak subtraction, based on resistance estimates from 4-5 hyperpolarizing pulses applied before the depolarizing test potential, was used for all voltage clamp recordings. Membrane currents were filtered at 2.5 kHz and sampled at 10 kHz. The pipette solution contained (in mM): 140 CsF, 1 EGTA, 10 NaCl and 10 HEPES (pH 7.3). The standard bathing solution was (in mM): 140 sodium chloride, 3 potassium chloride, 1 magnesium chloride, 1 calcium chloride, and 10 HEPES (pH 7.3). The liquid junction potential for these solutions is <8 mV; data was not corrected to account for this offset. The osmolarity of all solutions was adjusted to 310 mOsm (Wescor 5500 osmometer). The offset potential was zeroed before patching the cells and checked after each recording for drift; if the drift was greater than 10 mV per hour, the recording was discarded.

There was no detectable TTX-R current, attributable to rNaN channels, in sixteen transfected cells, although other currents could be detected. Thus, although the pLG338XM-rNaN construct is translated into protein in transfected HEK293 cells, it does not produce detectable rat NaN/SNS2/Naᵥ1.9 currents.

### EXAMPLE 6

Development of a Non-Mutated Rat Naᵥ1.9 Expression Vehicle.

The non-mutated (at the amino acid level) and non-expressing rNaN clone pLG338-rNaN2 served as the basis for the construction of a non-mutated expression vector for rNaN. Polyadenylation (polyA) sequences, followed by a neomycin resistance gene, were inserted into pLG338-rNaN2 3′ of the rNaN open reading frame.

Briefly, pLG338-rNaN2 was digested with NotI and XbaI and the following SalI linker (JDG08 (SEQ ID NO: 11) and JDG09 (SEQ ID NO: 12)), with NotI/XbaI overhangs, was inserted into the plasmid. Both the NotI and XbaI sites were retained.

```
(5XD4F6)
5′-cagagaccaagcagctcactagc-3′
(JDG23)
                NotI ClaI
5′-aaagcggccgcatcgattatcagtcattgtgaaccttgac-3′
                                      2x stop
```

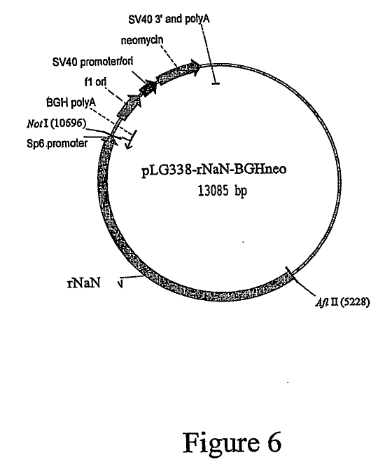

The plasmid Rc/CMV was digested with NotI, SalI and PvuI and the approximately 2.4 kb BGH polyA, SV40 promoter, neomycin resistance gene, and SV40 polyA containing fragment was gel purified and cloned into pLG338-rNaN2 which had been digested with NotI and SalI, and gel purified. This clone (pLG338-rNaN-BGHneo, FIG. 6) was stable by restriction analysis.

GFP fused to the carboxy terminus of the rNaN orf would be desirable as a means of identifying mammalian cells that had been transfected. Thus, a rNaN-GFP fusion construct was created in two steps. First, the 3′ rNaN sequences in pLG338-rNaN-BGHneo were replaced by a PCR product that eliminated the rNaN stop codon and UTR sequences, and introduced a ClaI site just 5′ of the NotI site in the vector. Next, six tandem histidine residues [His(6)], followed by GFP was inserted at the 3′ end of rNaN. The six histidine residues were incorporated to provide a spacer between the rNaN and GFP open reading frames, and to provide a means of purifying the fusion protein on a nickel column.

Briefly, pLG338-rNaN2-BGHneo was used as template in a PCR reaction with the 5′ primer (3AP2F2; SEQ ID NO: 13) that annealing upstream of the unique Bsu36I site in rNaN, and a 3′ primer (JDG13; SEQ ID NO: 14) annealing to the final nineteen nucleotides of rNaN (but not the TGA stop codon) and engineered NotI and ClaI restriction enzyme sites at the 3′ end of the rNaN ORF.

| | |
|---|---|
| | SalI |
| (JDG08) | 5′-ggccgcaaagtcgact-3′ |
| (JDG09) | 5′-cgtttcagctgagatc-3′ |

The product of this PCR amplification was digested with Bsu36I and NotI, gel purified, and used to replace the corresponding Bsu36I/NotI sequences in pLG338-rNaN2-BGHneo. GFP was PCR amplified from the plasmid pEGFP-C2 (Clontech) using the JDG14 (SEQ ID NO: 15) and JDG15 (SEQ ID NO: 16) primers.

| | |
|---|---|
| | ClaI His (6) 5′ GFP |
| (JDG14) | 5′-aaaatcgatcatcatcatcatcatcatatggtgagcaagggcgagg-3′ |
| NotI stop 3′ GFP | |
| (JDG15) | 5′-aaaagcggccgcctacttgtacagctcgtccatg-3′ |

PCR reactions for both amplifications were: 35 cycles of 95° C. one minute, 55° C. one minute, 72° C. three minutes using Platinum PCR Supermix, 1 ng template, 0.2 μM each oligo primer and 1 μl (2.5 units) Pfu polymerase.

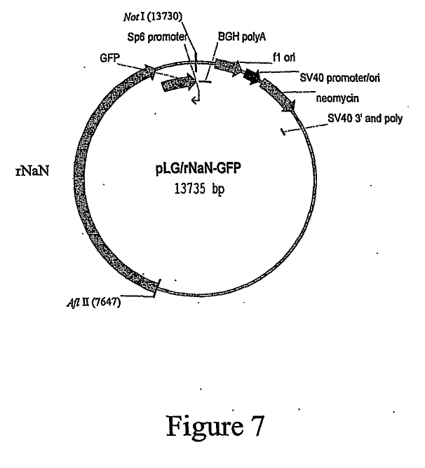

The GFP PCR product was digested with ClaI and NotI, gel purified, and cloned into the rNaN construct which had also been digested ClaI/NotI and gel purified. This resulted in a promotorless plasmid that has a rNaN-Ile-Asp-His(6)-GFP fusion followed by BGH polyA and neomycin resistance sequences. This plasmid (pLG/rNaN-GFP) has AflII and NotI sites flanking the rat Naᵥ1.9-His(6)-GFP fusion (FIG. 7).

Since an expression version of the low copy number pLG338 based plasmid (pLG338 XM, FIG. 2) had already been constructed it was decided to attempt to subclone the AflII/NotI fragment from pLG/rNaN-GFP into that expression vector. Thus, both clones were digested with AflII/NotI, the appropriate fragments gel purified and ligated together.

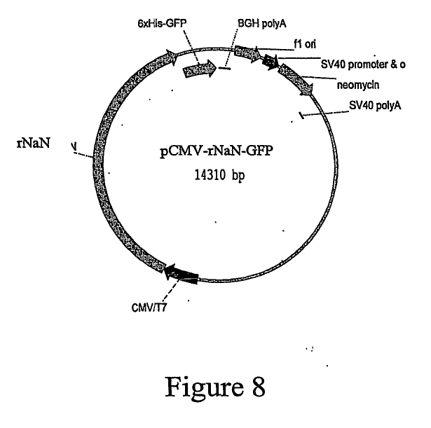

Colonies were screened by restriction analysis and one of those that appeared stable was sequenced and confirmed to be unchanged from the published rNaN sequence at the amino acid level (pCMV-rNaN-GFP, FIG. 8).

Stability of this clone can be attributed to the combination of plasmid and gene sequences used in this construct, the presence of the His(6)-GFP sequences exerting a stabilizing affect on the construct, and the removal of the 3′ untranslated sequences.

### EXAMPLE 7

Electrophysiological Recordings of TTX-R Currents from pCMV-rNaN-GFP

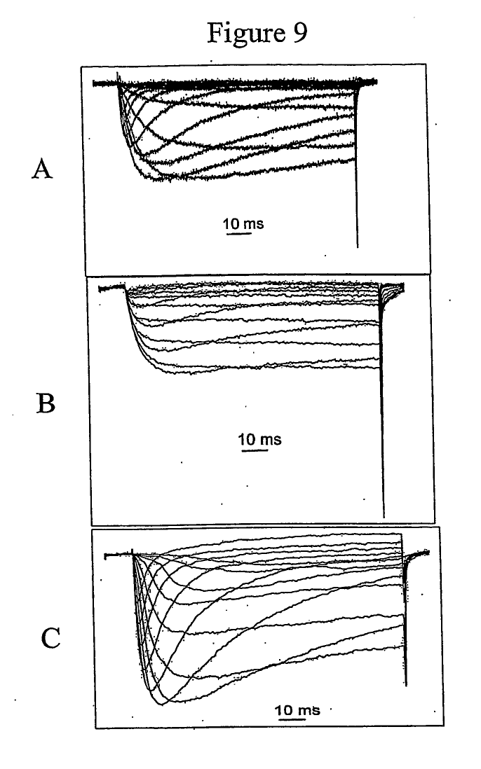

Using the method described in Example 5, electrophysiological measurements were taken from cells transfected with pCMV-rNaN-GFP and compared with normal rat DRG neurons. As shown in FIG. 9, the transfected HEK293 cells and mouse SNS-null DRG neurons had the same current signature as normal rat DRG neurons.

### EXAMPLE 8

rNaᵥ1.9 Expression Vector without His(6)-GFP

Construction of an expression vector consisting of 38 nucleotides of untranslated sequence 5′ of the rNaN atg initiation codon, followed by the entire rNaN ORF with translation termination signals (stop codons) at the end of the ORF. A non-fusion form of rNaN was constructed for expression that contained stop codons at the carboxy terminal end of rNaN. The following strategy was used to accomplish this goal.

Briefly, 3′ rNaN sequences were PCR amplified with the primers 5XD4F6 and JDG23. 5XD4F6 (SEQ ID NO: 17) is a sequencing primer that anneals upstream (5′) of the unique BglII site in rNaN. JDG23 (SEQ ID NO: 18) is a reverse primer that anneals to the last six amino acids (18 nucleotides) of rNaN and engineers two stop codons (TGATAA) followed by BspDI (isoschizomer of ClaI) and NotI sites at the 5′ end. PCR reactions were performed as previously described above.

(3AP2F2)
5′-gccttcgtggtcatctttacc-3′
(JDG13)
NotI ClaI
5′-aaaagcggccgctttatcgatgtcattgtgaaccttcacc-3′

The product of this PCR was used to replace the BglII/NotI fragment in pCMV-rNaN-GFP, resulting in the elimination of the His(6)-GFP sequence and the placement of two stop codons, followed by BspDI and NotI restriction enzyme sites at the carboxy terminus of the rNaN open reading frame.

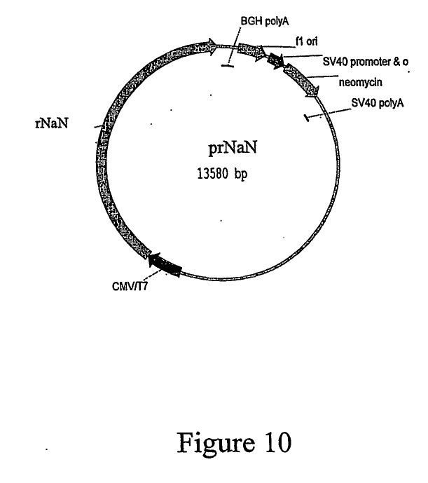

The plasmid (prNaN, FIG. 10) resulting from this series of cloning experiments appeared by restriction analysis to be correct and stable, and was submitted for sequencing. Sequencing results show the rNaN sequences to be identical to that of the published sequence at the amino acid level.

### EXAMPLE 9

Method of Measuring Sodium Current Flow

Sodium currents are measured in transfected HEK293 cells using patch clamp methods (Hamill et al. (1981) Pflügers Arch. 391, 85-100), as described by Rizzo et al. (1994) J. Neurophysiology 72, 2796-2815 and Dib-Hajj et al. (1997) FEBS Letters 416, 11-14). For these recordings data are acquired on a Macintosh Quadra 950 or similar computer, using a program such as Pulse (v 7.52) (HEKA). Fire polished electrodes (0.8-1.5 MW) are typically fabricated from capillary glass using a Sutter P-87 puller or a similar instrument. In the most rigorous analyses, cells are usually only considered for analysis if initial seal resistance is <5 GΩ, they have high leakage currents (holding current <0.1 nA at −80 mV), membrane blebs, and an access resistance <5 MΩ. Access resistance is usually monitored throughout the experiment and data are not used if resistance changes occur. Voltage errors are minimized using series resistance compensation and the capacitance artifact is canceled using computer controlled amplifier circuitry or other similar methods. For comparisons of the voltage dependence of activation and inactivation, cells with a maximum voltage error of ±10 mV after compensation are used. Linear leak subtraction is usually used for voltage clamp recordings. Membrane currents are typically filtered at 5 KHz and sampled at 20 KHz. The pipette solution contains a standard solution such as: 140 mM CsF, 2 mM magnesium chloride, 1 mM EGTA, and 10 mM Na-HEPES (pH 7.3). The standard bathing solution is usually 140 nM sodium chloride, 3 mM potassium chloride, 2 mM magnesium chloride, 1 mM calcium chloride, 10 mM HEPES and 10 mM glucose (pH 7.3).

Voltage clamp studies on transfected cells or DRG neurons, using methods such as intracellular patch-clamp recordings, can provide a quantitative measure of the sodium current density (and thus the number of sodium channels in a cell), and channel physiological properties. These techniques, which measure the currents that flow through ion channels such as sodium channels, are described in Rizzo et al. (1995) Neurobiol. Dis. 2, 87-89. Alternatively, the blockage or enhancement of sodium channel function can be measured using optical imaging with sodium-sensitive dyes or with isotopically labeled Na. These methods which are described by Rose et al. (1997) J Neurophysiol. 78, 3249-3258, measure the increase in intracellular concentration of sodium ions that occurs when sodium channels are open.

### EXAMPLE 10

Measurement of Intracellular Sodium ([Na⁺]ᵢ)

The effects of various agents on cells that express Na⁺ can be determined using ratiometric imaging of [Na⁺]ᵢ using SBFI or other similar ion-sensitive dyes. In this method, as described by Sontheimer et al. (1994) J. Neurosci. 14, 2464-2475, cytosolic-free Na⁺ is measured using an indicator for Na⁺, such as SBFI (sodium-binding benzofuran isophthalate (Harootunian et al. (1989) J. Biol. Chem. 264, 19458-19467)) or a similar dye. Cells are first loaded with the membrane-permeable acetoxymethyl ester form of the dye (which is dissolved in dimethyl sulfoxide (DMSO) at a stock concentration of 10 mM). Recordings are obtained on the stage of a microscope using a ratiometric imaging setup (e.g., from Georgia Instruments). Excitation light is provided at appropriate wavelengths (e.g., 340/385 nm). Excitation light is passed to the cells through a dichroic reflector (400 nm) and emitted light above 450 nm is collected. Fluorescence signals are amplified, e.g., by an image intensifier (GenIISyS) and collected with a CCD camera, or similar device, interfaced to a frame grabber. To account for fluorescence rundown, the fluorescence ratio 340:385 is used to assay cytosolic free Na⁺.

For calibration of SBFI's fluorescence, cells are perfused with calibration solutions containing known Na⁺ concentrations (typically 0 and 30 mM, or 0, 30 and 50 mM [Na⁺]), and with ionophones such as gramicidin and monensin (see above) after each experiment. As reported by Rose and Ransom (1996) J. Physiol. 491, 291-305), the 345/390 nm fluorescence ratio of intracellular SBFI changes monotonically with changes in [Na⁺]ᵢ. Experiments are typically repeated on multiple (typically at least four) different coverslips, providing statistically significant measurements of intracellular sodium in control cells, and in cells exposed to various concentrations of agents that may block, inhibit or enhance sodium current.

### EXAMPLE 11

Method to Measure Na⁺ Influx Via Measuring ²²Na or ⁸⁶Rb.

²²Na is a gamma emitter and can be used to measure Na⁺ flux (Kimelberg and Waltz (1988), Boulton, Baker, and Walz, Eds.), and ⁸⁶Rb⁺ can be used to measure Na⁺/K⁺-ATPase activity (Sontheimer et al. (1994) J. Neurosci. 14, 2464-2475). ⁸⁶Rb⁺ ions are taken up by the Na⁺/K⁺-ATPase-like K⁺ ions, but have the advantage of a much longer half-life than ⁴²K⁺ (Kimelberg and Mayhew (1975) J. Biol. Chem. 250, 100-104). Thus, measurement of the unidirectional ouabain-sensitive ⁸⁶Rb⁺ uptake provides a quantitative method for assaying Na⁺/K⁺-ATPase activity that provides another indicator of the electrical firing of nerve cells. Following incubation of cells expressing Naᵥ1.9 with the isotope ²²Na⁺ the cellular content of the isotope is measured by liquid scintillation counting or a similar method, and cell protein is determined using a method such as the bicinchoninic acid protein assay (Smith et al. (1985) Anal. Biochem. 150, 76-85) following the modifications described by Goldschmidt & Kimelberg (1989) Analytical Biochemistry 177, 41-45 for cultured cells. ²²Na and ⁸⁶Rb⁺ fluxes are determined in the presence and absence of agents that may block, inhibit, or enhance Naᵥ1.9. This permits determination of the actions of these agents on Naᵥ1.9.

### EXAMPLE 12

Establishment of Stable Cell Line for Naᵥ1.9.

Using the standard calcium phosphate protocol (Watanabe et al. (1999) Neurosci. Res. 33, 71-78), HEK 293 cells (10 cm plate at 50% confluency) were transfected with 10 μg pCMV rNaN-2xStop. This construct encodes the full open reading frame of the channel, not fused to the GFP protein. After two days the cells were split into two dishes with selective medium (complete medium of DMEM/Ham's F12 with 10% FCS+G418, 0.6 mg/ml). The medium was replaced every four to five days. When isolated colonies arose they were plated onto 24 well plates and grown in the selective medium.

Total cellular RNA from stable cell lines expressing rNaᵥ1.9 was isolated using RNeasy mini-columns (Qiagen). First strand cDNA was reverse transcribed in a 50 μl final volume using 1 mM random hexamer (Roche), 500 units SuperScript II reverse transcriptase (Life Technologies) and 100 units of RNase Inhibitor (Roche). The buffer consisted of 50 mM Tris-HCl (pH 8.3), 75 mM KCl, 3 mM magnesium chloride, 10 mM DTT and 5 mM dNTP. The reaction was allowed to proceed at 37° C. for ninety minutes, 42° C. for thirty minutes and then terminated by heating to 95° C. for five minutes. Control templates were prepared in identical fashion except for the omission of the reverse transcriptase enzyme (−RT).

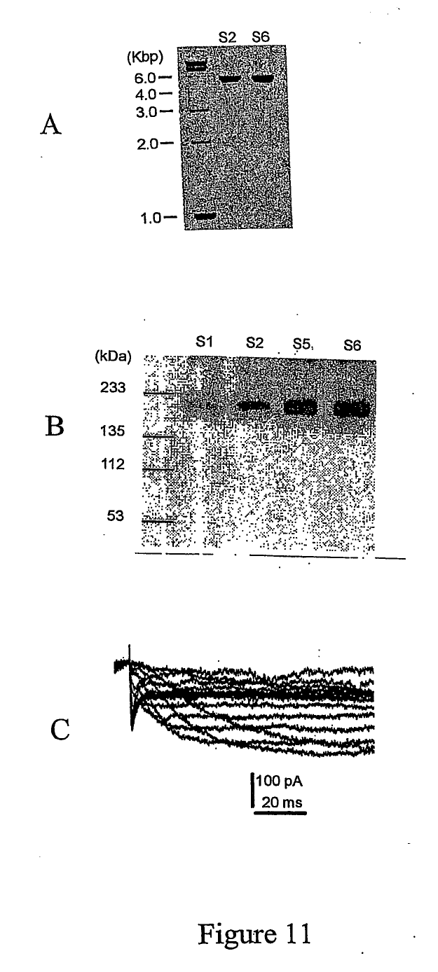

PCR amplification was carried out using primers which spanned bases 20-42 and 5335-5314 of the published rNaᵥ1.9 sequence for 35 cycles, annealing at 58° C. and extending for eight minutes. A single amplification product of the expected size of 5.3 kilobases was obtained from the selected clones (FIG. 11A). Control PCR reactions in which the −RT template was used produced no amplification products. Amplification was carried out a programmable thermal cycler (PTC-100, MJ Research).

Four HEK293 cell lines (S1, S2, S5 and S6) stably transfected with rNaᵥ1.9 cDNA were investigated for the production of a full length rNaᵥ1.9 channel protein. Cells were collected from 100 mm dishes by scraping and were homogenized in a glass dounce in ice cold lysis buffer at 30 μl/mg of tissue. The lysis buffer (0.3 M sucrose, 10 mM Tris (pH 8.1), 2 mM EDTA) was supplemented with protease inhibitors (1 mM PMSF, 10 μg/ml Aprotinin 10 μg/ml Leupeptin, mM DTT, 1 mM Benzamidine, 1 mM Pepstatin, 8 μg/ml Calpain I, 8 μg/ml Calpain II). Homogenates were kept on ice for one hour before centrifugation at 1000×g (low-speed spin) for seven minutes at 4° C. to remove nuclei and intact cells; The pellet was re-homogenized and spun again under the same conditions. The supernatants from the two low-speed spins were combined and centrifuged at 120,000×g for one hour at 4° C. The pellet, containing the total membrane fraction, was suspended in 0.2 M KCl, 10 mM HEPES (pH 7.4).

To solubilize the membrane fraction, an equal volume of 5% Triton x-100, 10 mM HEPES (pH 7.4) was added to the sample and the suspension was kept on ice for one hour. The unsolubilized material was pelleted by centrifugation at 10,000×g for ten minutes at 4° C. and the soluble material in the supernatant was collected for further processing. Protein content was determined using BioRad DC assay for high detergent samples.

An anti-Naᵥ1.9 polyclonal antibody was raised in rabbits against the C-terminal 18 amino acid peptide CNGDLSSLDVAKVKVHND (amino acids 1748-1765 of SEQ ID NO: 20), and affinity purified over the specific peptide-column (Fjell et al. (2000) Neuro report 11, 199-202). Anti-Naᵥ1.9 antibody was used at a final concentration of 0.2 μg/ml.

An immunoblot assay was done as previously described (Tyrrell et al. (2001) J. Neurosci. 21, 9629-9637). Briefly, samples (10-20 μg) were denatured in Laemmli sample buffer for twenty minutes at 37° C. Proteins were fractionated by SDS/PAGE using either 5%, or 4-15% gradient Tris-HCl Ready Gels (BioRad) and then electro-transferred to Immobilon-P membrane (Millipore) overnight at 22 mV and 4° C. Blots were blocked with 10% dried milk in TBS for one hour at room temperature before incubation for two hours at room temperature with the primary antibody diluted in 5% BSA in TBS. Blots were washed extensively in TBST (TBS plus 0.2% Tween-20). Immunoreactive proteins were detected by incubating with a 1:10,000 dilution in 1.25% BSA of a goat anti-rabbit IgG secondary antibody conjugated to horseradish peroxidase (Dako A/S) for one hour at room temperature. The signal was detected by Renaissance chemiluminescence according to manufacturer's recommendations (NEN). A robust immunoreactive protein of the expected size of approximately 210 kDa is detected in each of the four cell lines (FIG. 11B).

Whole-cell patch-clamp recordings using stably transfected cells were conducted at room temperature (21° C.) using an EPC-9 amplifier. Data was acquired on a Pentium-III computer using the Pulse program (v 8.31) (HEKA Electronic). Fire-polished electrodes (0.8-1.5 MΩ) were fabricated from 1.7-mm capillary glass (VWR) using a Sutter P-97 puller. Cells were not considered for analysis if the initial seal resistance was less than 2 GΩ or if they had an access resistance greater than 4 MΩ. Linear leak subtraction, based on resistance estimates from 4-5 hyperpolarizing pulses applied before the depolarizing test potential, was used for all voltage clamp recordings. Membrane currents were usually filtered at 2.5 kHz and sampled at 10 kHz. The pipette solution contained 140 mM CsF, 1 mM EGTA, 10 mM NaCl and 10 mM HEPES (pH 7.3). The standard bathing solution was 140 mM NaCl, 3 mM KCl, 1 mM magnesium chloride, 1 mM calcium chloride, and 10 mM HEPES (pH 7.3). Cadmium (100 μM) was added to the bath solution to block calcium currents and 500 nM tetrodotoxin was added to block endogenous tetrodotoxin-sensitive sodium currents. Osmolarity of all solutions was adjusted to around 310 mosM (Wescor 5500 osmometer). Persistent TTX-resistant sodium currents were displayed by stably expressed Naᵥ1.9 sodium channels in the S2 cell line (FIG. 11C) and were not observed in naive HEK293 cells (data not shown).

### EXAMPLE 13

Viral Expression Vector Comprising rNaᵥ1.9

Adenovirus Ad5 can be used to generate a viral expression vector for Naᵥ1.9. Ad5 early region 1 (E1) and early region 3 (E3) sequences are replaced with a CMV promoter followed by the cloned Naᵥ1.9 gene. HEK293 cells, which are E1-transcomplementing, are transfected with the Ad5-Naᵥ1.9 viral expression vector. Stably expressing cells are then selected using anti-Naᵥ1.9 antibody.

### EXAMPLE 14

Human and Murine Naᵥ1.9 Expression Constructs

Using the standard calcium phosphate protocol (Watanabe et al. (1999) Neurosci. Res. 33, 71-78) HEK 293 cells (10 cm plate at 70% confluency) were transfected separately with 10 μg of human and mouse pCMV-NaN. These constructs encode the full open reading frame of the channel. Cells were grown in complete medium of DMEM-Ham's F12 supplemented with 10% fetal calf serum.

Total cellular RNA from stable cell lines expressing NaN/Naᵥ1.9 was isolated using RNeasy mini-columns (Qiagen). First strand cDNA was reverse transcribed in a 50 μl final volume using 1.0 μM random hexamer (Roche), 500 units SuperScript II reverse transcriptase (Life Technologies) and 100 units of RNase Inhibitor (Roche). The buffer consisted of 50 mM Tris-HCl (pH 8.3), 75 mM KCl, 10 mM DTT, 3 mM MgCl₂ and 5 mM dNTP. The reaction was allowed to proceed at 37° C. for 90 min, 42° C. for thirty minutes and then terminated by heating to 95° C. for five minutes. Control templates were prepared in identical fashion except for the omission of the reverse transcriptase enzyme (−RT).

PCR amplification was carried out using 1 μM primers (Table 1) for 35 cycles, annealing at 58° C. and extending for one minute per kilobase. Control PCR reactions in which the −RT template was used produced no amplification products. Amplification was carried out in a PTC-100 programmable thermal cycler (MJ Research).

Anti-Naᵥ1.9 polyclonal antibodies were raised in rabbits against C-terminal peptides of human (CNGDLSSFGVAKGKVH, SEQ ID NO: 22), and mouse (CNGDLSSLDVAKVKVHND, SEQ ID NO: 23) Naᵥ1.9 channels, and affinity purified over the specific peptide-columns. Anti-Naᵥ1.9 antibodies were used at a final concentration of 2 μg/ml to stain HEK 293 cells transfected with human or mouse Naᵥ1.9 constructs.

TABLE 1

Primers with restriction sites at the 5′ tail of
the oligonucleotides are underlined

| | |
|---|---|
| Primers | Sequence |

| | |
|---|---|
| HuNaᵥ1.9Afl II | 5′acgccttaaggtgaagatggatgacagatg |
| | ct3′ |
| | (SEQ ID NO: 24) |
| HuNaᵥ1.9 Xba I | 5′ggttctagatcagtcacagtggaccttgc |
| | c3′ |
| | (SEQ ID NO: 25) |
| HuNaᵥ1.9-1950 | 5′gcctcggcgaaagtagtggtagg3′ |
| | (SEQ ID NO: 26) |
| HuNaᵥ1.9-1928 | 5′cctaccactactttcgccgagg3′ |
| | (SEQ ID NO: 27) |
| HuNaᵥ1.9-3897 | 5′tacgaagtaaatgtaaccgagtga3′ |
| | (SEQ ID NO: 28) |
| HuNaᵥ1.9-3877 | 5′ctcggttacatttacttcgtagt3′ |
| | (SEQ ID NO: 29) |
| MNaᵥ1.9Not1 | 5′tatatatgcggccgcaagatggaggagagg |
| | tactatccag3′ |
| | (SEQ ID NO: 30) |
| MNaᵥ1.9-1813 | 5′tattgtggtgttccatggccaaga3′ |
| | (SEQ ID NO: 31) |
| MNaᵥ1.9-1829 | 5′gccatggaacaccacaatatG3′ |
| | (SEQ ID NO: 32) |
| MNaᵥ1.9-3828 | 5′cacgaagtaaaggtatgcgtatag3′ |
| | (SEQ ID NO: 33) |
| MNaᵥ1.9-3747 | 5′tatgaatgcagctgttgattccag3′ |
| | (SEQ ID NO: 34) |
| MNaᵥ1.9Apa I/Pvu I | 5′gatcgatcgggccctcagtcacaatgaacc |
| | ttgatcttggg3′ |
| | (SEQ ID NO: 35) |

TABLE 2

Human and mouse Naᵥ1.9 amplification primers and
the predicted length of the amplicons

| | Amplicon | Amplicon |
|---|---|---|
| Primer Pair | coordinates | Length |
| (a) HuNaᵥ1.9Af1 II*/HuNav1.9XbaI | 24-5406 | 5400 |
| (b) HuNaᵥ1.9Af1 | 24-1950 | 1936 |
| II-HuNaᵥ1.9-1950 | | |
| (c) HuNaᵥ1.9-1928-HuNaᵥ1.9-3897 | 1928-3897 | 1970 |
| (d) HuNaᵥ1.9-3877-NuNav1.9Xba I | 3877-5406 | 1539 |
| (e) MNaᵥ1.9Not1-MNaᵥ1.9-1813 | 50-1847 | 1813 |
| (f) MNaᵥ1.9-1829-MNaᵥ1.9-3828 | 1829-3862 | 2034 |
| (g) MNaᵥ1.9-3747-MNaᵥ1.9Apa | 3781-5350 | 1584 |
| I/Pvu I | | |

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety for all purposes.

What is claimed is:

1. An expression vector comprising a nucleic acid sequence which encodes a mammalian Naᵥ1.9 sodium channel protein or a fragment thereof, wherein the expression vector produces a sodium current when transfected in a cell.

2. The expression vector of claim 1, wherein the mammalian Naᵥ1.9 is selected from the group consisting of human, rat and murine Naᵥ1.9.

3. The expression vector of claim 2, wherein the rat Naᵥ1.9 comprises an amino acid sequence selected from the group consisting of:
(a) amino acid residues 1 to 1765 of SEQ ID NO: 2,
(b) an amino acid sequence comprising a fragment of amino acid residues 1 to 1765 of SEQ ID NO: 2, and
(c) amino acid residues 1 to 1765 of SEQ ID NO: 2 comprising at least one conservative substitution.

4. The expression vector of claim 2, wherein the human Naᵥ1.9 comprises an amino acid sequence selected from the group consisting of:
(a) amino acid residues 1 to 1791 of SEQ ID NO: 4,
(b) an amino acid sequence comprising a fragment of amino acid residues 1 to 1791 of SEQ ID NO: 4, and
(c) amino acid residues 1 to 1791 of SEQ ID NO: 4 comprising at least one conservative substitution.

5. The expression vector of claim 2, wherein the murine Naᵥ1.9 comprises an amino acid sequence selected from the group consisting of:
(a) amino acid residues 1-1765 of SEQ ID NO: 6,
(b) an amino acid sequence comprising a fragment of amino acid residues 1-1765 of SEQ ID NO: 6, and
(c) amino acid residues 1-1765 of SEQ ID NO: 6 comprising at least one conservative substitution.

6. The expression vector of claim 1 that is an expression plasmid.

7. The expression plasmid of claim 6 that is a low copy number expression plasmid.

8. The expression plasmid of claim 7 further comprising a promoter sequence operably linked to the Naᵥ1.9 sequence.

9. The expression plasmid of claim 8, wherein the promoter sequence is a CMV promoter.

10. The expression plasmid of claim 8 further comprising a selectable marker under the control of a second promoter sequence.

11. The expression plasmid of claim 10, wherein the selectable marker is a neomycin resistance gene.

12. The expression plasmid of claim 7, wherein the Naᵥ1.9 is rat Naᵥ1.9 and wherein the rat Naᵥ1.9 comprises SEQ ID NO: 20.

13. The expression plasmid of claim 12, further comprising at the 5′ end adjacent to the open reading frame encoding said SEQ ID NO: 2 at least about 1-100 of the untranslated nucleic acid residues which are 5′ to the open reading frame of SEQ ID NO: 19.

14. The expression plasmid of claim 13, comprising at least about 1-50 of said 5′ untranslated nucleic acid residues.

15. The expression plasmid of claim 14, comprising 38 of the 5′ untranslated nucleic acid residues.

16. The expression plasmid of claim 15, wherein the expression plasmid is prNaN.

17. A recombinant cell comprising a plasmid selected from the group consisting of prNaN and a plasmid of claim 12.

18. The expression plasmid of claim 13, further comprising a nucleic acid sequence which encodes a histidine tag sequence wherein the expression of the tag is under the control of the same promoter which controls expression of the open reading frame.

19. The expression plasmid of claim 18, further comprising a nucleic acid sequence which encodes a green fluorescent protein (GFP) label polypeptide wherein the expression of the GFP is under the control of the same promoter which controls expression of the open reading frame.

20. The expression plasmid of claim 19, which is pCMV-rNaN-GFP.

21. A recombinant cell comprising a plasmid selected from the group consisting of pCMV-rNaN-GFP and a plasmid of claim 19.

22. An expression plasmid comprising a nucleic acid sequence which encodes a mammalian Naᵥ1.9 sodium channel protein or a fragment thereof, wherein the mammalian Naᵥ1.9 sodium channel protein or fragment thereof comprises SEQ ID NO: 2 with at least one mutation selected from the group consisting of: a serine to proline change at amino acid position 962, a leucine to proline change at amino acid position 1282 and a deletion of amino acid residues 1000-1010.

23. The expression plasmid of claim 22, wherein the expression plasmid is pLG338XM-rNaN.

24. A recombinant cell comprising a plasmid selected from the group consisting of pLG338XM-rNaN and a plasmid of claim 22.

25. A method of making a cell or cell line that produces a Naᵥ1.9 sodium channel-dependent sodium current comprising:
(a) providing a cell which has been transfected with an expression vector which comprises a nucleic acid sequence which encodes the Naᵥ1.9 sodium channel protein, and
(b) culturing said cell under conditions which allow expression of the Naᵥ1.9 sodium channel protein to produce a sodium current in the transfected cell.

26. The method of claim 25, wherein the mammalian Naᵥ1.9 is selected from the group consisting of human, rat and murine Naᵥ1.9.

27. The method of claim 26, wherein the expression vector is an expression plasmid.

28. The method of claim 27, wherein the expression plasmid is selected from the group consisting of prNaN, pCMV-rNaN-GFP and a plasmid comprising SEQ ID NO: 19.

29. A method of screening for an agent that modulates sodium current in a cell comprising:
(a) exposing the cell or cell line produced by the method of claim 25 to the agent; and
(b) measuring sodium current following exposure to the agent, wherein an alteration in the level of sodium current is indicative of an agent capable modulating sodium current in a cell.

30. A recombinant cell comprising the expression vector of claim 1.

31. The expression vector of claim 1, wherein the vector is a viral vector.

32. The viral vector of claim 31, wherein the viral vector is selected from the group consisting of adenovirus, adeno-associated virus and baculovirus.

33. A recombinant cell comprising the viral vector of claim 31.


SEQUENCE LISTING (35 sequences)

SEQ ID NO: 1 - DNA, 5905 bp, Rattus norvegicus
  CDS (52)..(5349)
tctggagccatacggtgccctgatcctctgtaccaggaagacagggtgaagatggaggagaggtactacccggtgatcttcccggacgagcggaatttccgccccttcacttccgactctctggctgccatagagaagcggattgctatccaaaaggagaggaagaagtccaaagacaaggcggcagctgagccccagcctcggcctcagcttgacctaaaggcctccaggaagttacctaagctttatggtgacattccccctgagcttgtagcgaagcctctggaagacctggacccattctacaaagaccataagacattcatggtgttgaacaagaagagaacaatttatcgcttcagcgccaagcgggccttgttcattctggggccttttaatcccctcagaagcttaatgattcgtatctctgtccattcagtctttagcatgttcatcatctgcacggtgatcatcaactgtatgttcatggcgaattctatggagagaagtttcgacaacgacattcccgaatacgtcttcattgggatttatattttagaagctgtgattaaaatattggcaagaggcttcattgtggatgagttttccttcctccgagatccgtggaactggctggacttcattgtcattggaacagcgatcgcaacttgttttccgggcagccaagtcaatctttcagctcttcgtaccttccgagtgttcagagctctgaaggcgatttcagttatctcaggtctgaaggtcatcgtaggtgccctgctgcgctcggtgaagaagctggtagacgtgatggtcctcactctcttctgcctcagcatctttgccctggtcggtcagcagctgttcatgggaattctgaaccagaagtgtattaagcacaactgtggccccaaccctgcatccaacaaggattgctttgaaaaggaaaaagatagcgaagacttcataatgtgtggtacctggctcggcagcagaccctgtcccaatggttctacgtgcgataaaaccacattgaacccagacaataattatacaaagtttgacaactttggctggtcctttctcgccatgttccgggttatgactcaagactcctgggagaggctttaccgacagatcctgcggacctctgggatctactttgtcttcttcttcgtggtggtcatcttcctgggctccttctacctgcttaacctaaccctggctgttgtcaccatggcttatgaagaacagaacagaaatgtagctgctgagacagaggccaaggagaaaatgtttcaggaagcccagcagctgttaagggaggagaaggaggctctggttgccatgggaattgacagaagttcccttaattcccttcaagcttcatccttttccccgaagaagaggaagtttttcggtagtaagacaagaaagtccttctttatgagagggtccaagacggcccaagcctcagcgtctgattcagaggacgatgcctctaaaaatccacagctccttgagcagaccaaacgactgtcccagaacttgccagtggatctctttgatgagcacgtggaccccctccacaggcagagagcgctgagcgctgtcagtatcttaaccatcaccatgcaggaacaagaaaaattccaggagccttgtttcccatgtgggaaaaatttggcctctaagtacctggtgtgggactgtagccctcagtggctgtgcataaagaaggtcctgcggaccatcatgacggatccctttactgagctggccatcaccatctgcatcatcatcaataccgttttcttagccgtggagcaccacaacatggatgacaacttaaagaccatactgaaaataggaaactgggttttcacgggaattttcatagcggaaatgtgtctcaagatcatcgcgctcgacccttaccactacttccggcacggctggaatgtttttgacagcatcgtggccctcctgagtctcgctgatgtgctctacaacacactgtctgataacaataggtctttcttggcttccctcagagtgctgagggtcttcaagttagccaaatcctggcccacgttaaacactctcattaagatcatcggccactccgtgggcgcgcttggaaacctgactgtggtcctgactatcgtggtcttcatcttttctgtggtgggcatgcggctcttcggcaccaagtttaacaagaccgcctacgccacccaggagcggcccaggcggcgctggcacatggataatttctaccactccttcctggtggtgttccgcatcctctgtggggaatggatcgagaacatgtggggctgcatgcaggatatggacggctccccgttgtgcatcattgtctttgtcctgataatggtgatcgggaagcttgtggtgcttaacctcttcattgccttgctgctcaattccttcagcaatgaggagaaggatgggagcctggaaggagagaccaggaaaaccaaagtgcagctagccctggatcggttccgccgggccttctccttcatgctgcacgctcttcagagtttttgttgcaagaaatgcaggaggaaaaactcgccaaagccaaaagagacaacagaaagctttgctggtgagaataaagactcaatcctcccggatgcgaggccctggaaggagtatgatacagacatggctttgtacactggacaggccggggctccgctggccccactcgcagaggtagaggacgatgtggaatattgtggtgaaggcggtgccctacccacctcacaacatagtgctggagttcaggccggtgacctccctccagagaccaagcagctcactagcccggatgaccaaggggttgaaatggaagtattttctgaagaagatctgcatttaagcatacagagtcctcgaaagaagtctgacgcagtgagcatgctctcggaatgcagcacaattgacctgaatgatatctttagaaatttacagaaaacagtttcccccaaaaagcagccagatagatgctttcccaagggccttagttgtcactttctatgccacaaaacagacaagagaaagtccccctgggtcctgtggtggaacattcggaaaacctgctaccaaatcgtgaagcacagctggtttgagagtttcataatctttgttattctgctgagcagtggagcgctgatatttgaagatgtcaatctccccagccggccccaagttgagaaattactaaggtgtaccgataatattttcacatttattttcctcctggaaatgatcctgaagtgggtggcctttggattccggaggtatttcaccagtgcctggtgctggcttgatttcctcattgtggtggtgtctgtgctcagtctcatgaatctaccaagcttgaagtccttccggactctgcgggccctgagacctctgcgggcgctgtcccagtttgaaggaatgaaggttgtcgtctacgccctgatcagcgccatacctgccattctcaatgtcttgctggtctgcctcattttctggctcgtattttgtatcttgggagtaaatttattttctgggaagtttggaaggtgcattaacgggacagacataaatatgtatttggattttaccgaagttccgaaccgaagccaatgtaacattagtaattactcgtggaaggtcccgcaggtcaactttgacaacgtggggaatgcctatctcgccctgctgcaagtggcaacctataagggctggctggaaatcatgaatgctgctgtcgattccagagagaaagacgagcagccggactttgaggcgaacctctacgcgtatctctactttgtggtttttatcatcttcggctccttctttaccctgaacctctttatcggtgttattattgacaacttcaatcagcagcagaaaaagttaggtggccaagacatttttatgacagaagaacagaagaaatattacaatgcaatgaaaaagttaggaaccaagaaacctcaaaagcccatcccaaggcccctgaacaaatgtcaagcctttgtgttcgacctggtcacaagccaggtctttgacgtcatcattctgggtcttattgtcttaaatatgattatcatgatggctgaatctgccgaccagcccaaagatgtgaagaaaacctttgatatcctcaacatagccttcgtggtcatctttaccatagagtgtctcatcaaagtctttgctttgaggcaacactacttcaccaatggctggaacttatttgattgtgtggtcgtggttctttctatcattagtaccctggtttcccgcttggaggacagtgacatttctttcccgcccacgctcttcagagtcgtccgcttggctcggattggtcgaatcctcaggctggtccgggctgcccggggaatcaggaccctcctctttgctttgatgatgtctctcccctctctcttcaacatcggtctgctgctcttcctggtgatgttcatttacgccatctttgggatgagctggttttccaaagtgaagaagggctccgggatcgacgacatcttcaacttcgagacctttacgggcagcatgctgtgcctcttccagataaccacttcggctggctgggataccctcctcaaccccatgctggaggcaaaagaacactgcaactcctcctcccaagacagctgtcagcagccgcagatagccgtcgtctacttcgtcagttacatcatcatctccttcctcatcgtggtcaacatgtacatcgctgtgatcctcgagaacttcaacacagccacggaggagagcgaggaccctctgggagaggacgactttgaaatcttctatgaggtctgggagaagtttgaccccgaggcgtcgcagttcatccagtattcggccctctctgactttgcggacgccctgccggagccgttgcgtgtggccaagccgaataagtttcagtttctagtgatggacttgcccatggtgatgggcgaccgcctccattgcatggatgttctctttgctttcactaccagggtcctcggggactccagcggcttggataccatgaaaaccatgatggaggagaagtttatggaggccaacccttttaagaagctctacgagcccatagtcaccaccaccaagaggaaggaggaggagcaaggcgccgccgtcatccagagggcctaccggaaacacatggagaagatggtcaaactgaggctgaaggacaggtcaagttcatcgcaccaggtgttttgcaatggagacttgtccagcttggatgtggccaaggtcaaggttcacaatgactgaaccctcatctccacccctacctcactgcctcacagcttagcctccagcctctggcgagcaggcggcagactcactgaacacaggccgttcgatctgtgtttttggctgaacgaggtgacaggttggcgtccatttttaaatgactcttggaaagatttcatgtagagagatgttagaagggactgcaaaggacaccgaccataacggaaggcctggaggacagtccaacttacataaagatgagaaacaagaaggaaagatcccaggaaaacttcagattgtgttctcagtacattccccaatgtgtctgttcggtgttttgagtatgtgacctgccacatgtagctcttttttgcatgtacgtcaaaaccctgcagtaagttaatagcttgctacgggtgttcctaccagcatcacagaattgggtgtatgactcaaacctaaaagcatgactctgacttgtcagtcagcaccccgactttcagacgctccaatctctgtcccaggtgtctaacgaataaataggtaaaagaaaaaaaaaaaaaaaaaaaaaaaa

SEQ ID NO: 2 - protein, 1765 aa, Rattus norvegicus
MEERYYPVIFPDERNFRPFTSDSLAAIEKRIAIQKERKKSKDKAAAEPQPRPQLDLKASRKLPKLYGDIPPELVAKPLEDLDPFYKDHKTFMVLNKKRTIYRFSAKRALFILGPFNPLRSLMIRISVHSVFSMFIICTVIINCMFMANSMERSFDNDIPEYVFIGIYILEAVIKILARGFIVDEFSFLRDPWNWLDFIVIGTAIATCFPGSQVNLSALRTFRVFRALKAISVISGLKVIVGALLRSVKKLVDVMVLTLFCLSIFALVGQQLFMGILNQKCIKHNCGPNPASNKDCFEKEKDSEDFIMCGTWLGSRPCPNGSTCDKTTLNPDNNYTKFDNFGWSFLAMFRVMTQDSWERLYRQILRTSGIYFVFFFVVVIFLGSFYLLNLTLAVVTMAYEEQNRNVAAETEAKEKMFQEAQQLLREEKEALVAMGIDRSSLNSLQASSFSPKKRKFFGSKTRKSFFMRGSKTAQASASDSEDDASKNPQLLEQTKRLSQNLPVDLFDEHVDPLHRQRALSAVSILTITMQEQEKFQEPCFPCGKNLASKYLVWDCSPQWLCIKKVLRTIMTDPFTELAITICIIINTVFLAVEHHNMDDNLKTILKIGNWVFTGIFIAEMCLKIIALDPYHYFRHGWNVFDSIVALLSLADVLYNTLSDNNRSFLASLRVLRVFKLAKSWPTLNTLIKIIGHSVGALGNLTVVLTIVVFIFSVVGMRLFGTKFNKTAYATQERPRRRWHMDNFYHSFLVVFRILCGEWIENMWGCMQDMDGSPLCIIVFVLIMVIGKLVVLNLFIALLLNSFSNEEKDGSLEGETRKTKVQLALDRFRRAFSFMLHALQSFCCKKCRRKNSPKPKETTESFAGENKDSILPDARPWKEYDTDMALYTGQAGAPLAPLAEVEDDVEYCGEGGALPTSQHSAGVQAGDLPPETKQLTSPDDQGVEMEVFSEEDLHLSIQSPRKKSDAVSMLSECSTIDLNDIFRNLQKTVSPKKQPDRCFPKGLSCHFLCHKTDKRKSPWVLWWNIRKTCYQIVKHSWFESFIIFVILLSSGALIFEDVNLPSRPQVEKLLRCTDNIFTFIFLLEMILKWVAFGFRRYFTSAWCWLDFLIVVVSVLSLMNLPSLKSFRTLRALRPLRALSQFEGMKVVVYALISAIPAILNVLLVCLIFWLVFCILGVNLFSGKFGRCINGTDINMYLDFTEVPNRSQCNISNYSWKVPQVNFDNVGNAYLALLQVATYKGWLEIMNAAVDSREKDEQPDFEANLYAYLYFVVFIIFGSFFTLNLFIGVIIDNFNQQQKKLGGQDIFMTEEQKKYYNAMKKLGTKKPQKPIPRPLNKCQAFVFDLVTSQVFDVIILGLIVLNMIIMMAESADQPKDVKKTFDILNIAFVVIFTIECLIKVFALRQHYFTNGWNLFDCVVVVLSIISTLVSRLEDSDISFPPTLFRVVRLARIGRILRLVRAARGIRTLLFALMMSLPSLFNIGLLLFLVMFIYAIFGMSWFSKVKKGSGIDDIFNFETFTGSMLCLFQITTSAGWDTLLNPMLEAKEHCNSSSQDSCQQPQIAVVYFVSYIIISFLIVVNMYIAVILENFNTATEESEDPLGEDDFEIFYEVWEKFDPEASQFIQYSALSDFADALPEPLRVAKPNKFQFLVMDLPMVMGDRLHCMDVLFAFTTRVLGDSSGLDTMKTMMEEKFMEANPFKKLYEPIVTTTKRKEEEQGAAVIQRAYRKHMEKMVKLRLKDRSSSSHQVFCNGDLSSLDVAKVKVHND

SEQ ID NO: 3 - DNA, 5860 bp, Homo sapiens
  CDS (31)..(5403)
atctgctcaagccaggaatctcgggtgaagatggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactgagccctcacctccacgcctacctcatagcttcacagccttgccttcagcctctgagctccaggggtcagcagcttagtgtatcaacagggagtggattcaccaaattagccattccattttcttttctggctaaaataaatgatatttcaatttcattttaaatgatacttacagagatataagataaggctacttgacaaccagtggtactattataataaggaagaagacaccaggaaggactgtaaaaggacataccaattttaggattgaaatagttcaggccgggcgcagtggctcatgcctgtaatcccagcactttgagaggccaaggcaggtggatcacgaggtcaagagatcgagaccatcctggccaacatgatgaaactccgtctctctaaaaatacaaaaattagctgggcatggtggcgtgcgcctgtagtcccactacttg

SEQ ID NO: 4 - protein, 1791 aa, Homo sapiens
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQAYELHQENKKPTSQRVQSVEIDMFSEDEPHLTIQDPRKKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 5 - DNA, 5822 bp, Mus musculus
  CDS (19)..(5313)
  misc_feature (5804)..(5804): n = a or t or c or g
tctgagccaagggtgaagatggaggagaggtactatccagtgatcttcccagacgagaggaatttccgccccttcactttcgactctttggctgcaatagagaagcggatcaccatccaaaaggagaagaagaaatccaaagacaaggcagcaactgagccccagcctcggcctcagctcgacctaaaggcctccaggaagttacctaagctctatggcgacgttccccctgaccttatagcgaagcccctggaagatctggacccattttacaaagaccataagacattcatggtattgaacaagaagagaacaatctatcgcttcagcgccaagagggccttgttcattctggggccttttaatcccatcagaagcttcatgattcgcatctctgtccattcagtcttcagcatgttcattatctgcacagtgatcatcaactgtatgttcatggctaataattcttctgtggacagtcgtcctagcagtaacattcccgaatacgtcttcattgggatttatgttttagaagctgtgattaaaatattggcaagaggcttcattgtggatgagttttcctacctccgagatccttggaactggctggacttcattgtcatcggaacagcgatagcgccttgttttctcggtaacaaagtcaataatctttccactctacgtaccttccgagtgttgagagctctgaaagccatttctgtaatctcaggtctgaaggtcatcgtgggtgccctgctgcgctccgtgaagaagctagtggacgtgatggtcctcactctcttttgcctcagcatctttgccctggttggtcagcagctcttcatgggaattctgagccagaaatgtattaaggacgactgtggccctaacgctttttccaacaaggattgctttgtaaaagaaaatgatagcgaggacttcataatgtgtggcaactggctcggcagaagatcctgccccgatggttccacgtgcaataaaaccacatttaacccagattataattatacaaactttgacagctttggctggtcttttctcgccatgttccgggttatgactcaagactcctgggagaagctttatcgacagatccttcgcacctccgggatctactttgtcttcttcttcgtggtcgtcatcttcctgggctctttctacctgcttaacttaaccctggctgtcgtcaccatggcttacgaggaacagaacagaaatgtcgctgccgagacagaggccaaggagaagatgtttcaggaagcccagcagctgttgagggaggaaaaggaggctctggttgccatgggaattgacagaacttcccttaattccctccaagcttcgtccttttccccaaagaagaggaagttttttggcagtaagacaagaaagtccttctttatgagagggtccaagacagcccgagcctcagcgtccgattcagaggacgatgcctctaaaaacccacaactccttgagcaaacaaaacgactatcccagaacttgcccgtagaactctttgatgagcacgtggaccccctccataggcagagagcgctgagtgccgtcagtatcttaaccatcaccatgcaggaacaagaaaaatcccaggagccttgtttcccgtgtgggaaaaacttggcatccaagtacctggtgtgggaatgtagccctccgtggctgtgcataaagaaggtcctgcagactatcatgacagaccccttcactgagctggccatcaccatctgcatcatcgtcaatactgtcttcttggccatggaacaccacaatatggataactctttaaaagacatactgaaaataggaaactgggttttcactggaattttcatagcggaaatgtgtctcaagatcattgcgctagacccttaccactacttccggcacggctggaacatctttgacagcattgtggcccttgtgagtctcgctgacgtgctcttccacaaactgtctaaaaacctctccttcttggcttccctcagagtgctgagggtcttcaagttagccaaatcctggcccacattaaacactctcattaagatcatcggccactccgtgggtgcgctcggaaacctgactgtggtcctaacgatcgtggtcttcatcttttccgtggttggcatgcggctctttggtgccaagtttaacaagacttgctccacctctccggagtccctccggcgctggcacatgggtgatttctaccattccttcctggtggtgttccgcatcctctgtggggagtggatcgagaacatgtgggaatgcatgcaggagatggaaggctccccgctgtgtgtcatcgtctttgtgctgatcatggtggtcgggaagctcgtggtgcttaacctcttcattgccttgctgctcaattccttcagcaatgaggaaaaggatgggaacccagaaggagagaccaggaaaaccaaagtgcagctagccctggatcggttcagccgagcgttctacttcatggcgcgcgctcttcagaatttctgttgcaagagatgcaggaggcaaaactcgccaaagccaaatgaggcaacagaaagctttgctggtgagagtagagacacagccaccctggatacaaggtcctggaaggagtatgattcagaaatgactctgtacactgggcaggccggggctccactggccccactggcaaaagaagaggacgatatggaatgttgtggtgaatgtgatgcctcacctacctcacagcctagtgaggaagctcaggcctgtgacctccctctgaagaccaagcggctccccagcccagatgaccacggggttgaaatggaagtgttttccgaagaagatccgaatttaaccatacagagtgctcgaaagaagtctgatgcggcaagcatgctctcagaatgcagcacaatagacctgaatgatatctttagaaatttacagaaaacagtttccccccaaaagcaaccagatcgatgctttcccaagggcctcagttgtatctttctatgttgcaaaacaatcaaaaaaaagtccccctgggtcctgtggtggaatcttcggaaaacctgctaccaaatcgtgaagcatagctggtttgagagcttcataatttttgtcatcctgctgagcagcggagcactgatattcgaagatgtcaatcttcccagccggccccaagttgaaaaattactgaagtgtaccgataatattttcacatttatttttctcctggaaatgattttgaagtgggtggcctttggattccggaagtatttcaccagtgcctggtgctggctcgatttcctcattgtggtggtgtctgtgctcagcctcacgaacttaccaaacttgaagtccttccggaatctgcgagcgctgagacctctgcgggcactgtctcagtttgaaggaatgaaggttgttgtcaatgccctcatgagtgccatacctgccatcctcaatgtcttgctggtctgcctcattttctggctcatattttgtatcctgggagtaaattttttttctgggaagtttggaagatgcattaatggaacagacataaataaatatttcaacgcttccaatgttccaaaccaaagccaatgtttagttagtaattacacgtggaaagtcccgaatgtcaactttgacaacgtggggaatgcctaccttgccctgctgcaagtggcgacctataagggctggctggacattatgaatgcagctgttgattccagagggaaagatgagcagccggcctttgaggcgaatctatacgcatacctttacttcgtggtttttatcatcttcggctcattctttaccctgaacctctttatcggtgttattattgacaacttcaatcagcagcagaaaaagttaggtggccaagacatttttatgacagaagaacagaagaaatattacaatgcaatgaaaaagttaggaaccaagaagcctcaaaagcccatcccaaggcccctgaacaaatgtcaagccttcgtgttcgatttggtcacaagccaggtctttgacgtcatcattctgggtcttattgtcacaaacatgattatcatgatggctgaatctgaaggccagcccaacgaagtgaagaaaatctttgatattctcaacatagtcttcgtggtcatctttaccgtagagtgtctcatcaaagtctttgctttgaggcaacactacttcaccaatggctggaacttatttgattgtgtggtcgtggttctttccatcattagtaccttggtttctggcttggagaacagcaacgtcttcccgcccacactcttcaggattgtccgcttggctcggatcggtcgaatcctcagactggtccgggcggctcgaggaatcaggacactccttttcgcgttgatgatgtctctcccctctctcttcaacattggtctgcttctctttctggtgatgttcatttatgccatctttgggatgaactggttttccaaagtgaagagaggctctgggattgatgacatcttcaactttgacactttctcgggcagcatgctctgcctcttccagataaccacttcagccggctgggatgctctcctcaaccccatgctggaatcaaaagcctcttgcaattcctcctcccaagagagctgtcagcagccgcagatagccatagtctacttcgtcagctacatcatcatctcctttctcattgtggttaacatgtacatagctgtgattctagagaacttcaacacagccacagaggagagcgaggaccccctgggcgaagacgactttgagatcttctatgagatctgggagaagtttgaccccgaagcaacacagttcatccagtactcatccctctctgacttcgccgacgccctgcccgagccgttgcgtgtggccaagcccaacaggtttcagtttctcatgatggacttgcccatggtgatgggtgatcgcctccattgcatggatgttctctttgctttcaccaccagggtcctcgggaactccagcggcttggataccatgaaagccatgatggaggagaagttcatggaggccaatcctttcaagaagttgtacgagcccattgtcaccaccacaaagaggaaggaggaggaggaatgtgccgctgtcatccagagggcctaccggagacacatggagaagatgatcaagctgaagctgaaaggcaggtcaagttcatcgctccaggtgttttgcaatggagacttgtctagcttggatgtgcccaagatcaaggttcattgtgactgaaacccccacctgcacgcctacctcacagcctcacagctcagcccccagcctctggcgaacaagcggcggactcaccgaacaggccgttcaacttgtttttttgggtgaaagaggtgataggttggtgtccatttttaaatgattcttggaaagattgaacgtcggaacatgttagaaaggactgccaaggacatccacagtaacggaaggcctgaaggacagttcaaattatgtaaagaaacgagaaggaaaggtcacatgtctgttcagttttaagtatgtgacctgccacatgtagctcctttgcatgttaagtgagaagtcaaaaccctgccataagtaaatagctttgttgcaggtgtttctaccagtgctgcggatttgggtgtatggctcaaacctgaaagcatgactctgacttgtcagcaccccaactttcagaagctctgatctctgtcctaggtgtttgacaaataaatacataaaanaaaaaaaaaaaaaaaaaa

SEQ ID NO: 6 - protein, 1765 aa, Mus musculus
  misc_feature (5804)..(5804): n = a or t or c or g
MEERYYPVIFPDERNFRPFTFDSLAAIEKRITIQKEKKKSKDKAATEPQPRPQLDLKASRKLPKLYGDVPPDLIAKPLEDLDPFYKDHKTFMVLNKKRTIYRFSAKRALFILGPFNPIRSFMIRISVHSVFSMFIICTVIINCMFMANNSSVDSRPSSNIPEYVFIGIYVLEAVIKILARGFIVDEFSYLRDPWNWLDFIVIGTAIAPCFLGNKVNNLSTLRTFRVLRALKAISVISGLKVIVGALLRSVKKLVDVMVLTLFCLSIFALVGQQLFMGILSQKCIKDDCGPNAFSNKDCFVKENDSEDFIMCGNWLGRRSCPDGSTCNKTTFNPDYNYTNFDSFGWSFLAMFRVMTQDSWEKLYRQILRTSGIYFVFFFVVVIFLGSFYLLNLTLAVVTMAYEEQNRNVAAETEAKEKMFQEAQQLLREEKEALVAMGIDRTSLNSLQASSFSPKKRKFFGSKTRKSFFMRGSKTARASASDSEDDASKNPQLLEQTKRLSQNLPVELFDEHVDPLHRQRALSAVSILTITMQEQEKSQEPCFPCGKNLASKYLVWECSPPWLCIKKVLQTIMTDPFTELAITICIIVNTVFLAMEHHNMDNSLKDILKIGNWVFTGIFIAEMCLKIIALDPYHYFRHGWNIFDSIVALVSLADVLFHKLSKNLSFLASLRVLRVFKLAKSWPTLNTLIKIIGHSVGALGNLTVVLTIVVFIFSVVGMRLFGAKFNKTCSTSPESLRRWHMGDFYHSFLVVFRILCGEWIENMWECMQEMEGSPLCVIVFVLIMVVGKLVVLNLFIALLLNSFSNEEKDGNPEGETRKTKVQLALDRFSRAFYFMARALQNFCCKRCRRQNSPKPNEATESFAGESRDTATLDTRSWKEYDSEMTLYTGQAGAPLAPLAKEEDDMECCGECDASPTSQPSEEAQACDLPLKTKRLPSPDDHGVEMEVFSEEDPNLTIQSARKKSDAASMLSECSTIDLNDIFRNLQKTVSPQKQPDRCFPKGLSCIFLCCKTIKKKSPWVLWWNLRKTCYQIVKHSWFESFIIFVILLSSGALIFEDVNLPSRPQVEKLLKCTDNIFTFIFLLEMILKWVAFGFRKYFTSAWCWLDFLIVVVSVLSLTNLPNLKSFRNLRALRPLRALSQFEGMKVVVNALMSAIPAILNVLLVCLIFWLIFCILGVNFFSGKFGRCINGTDINKYFNASNVPNQSQCLVSNYTWKVPNVNFDNVGNAYLALLQVATYKGWLDIMNAAVDSRGKDEQPAFEANLYAYLYFVVFIIFGSFFTLNLFIGVIIDNFNQQQKKLGGQDIFMTEEQKKYYNAMKKLGTKKPQKPIPRPLNKCQAFVFDLVTSQVFDVIILGLIVTNMIIMMAESEGQPNEVKKIFDILNIVFVVIFTVECLIKVFALRQHYFTNGWNLFDCVVVVLSIISTLVSGLENSNVFPPTLFRIVRLARIGRILRLVRAARGIRTLLFALMMSLPSLFNIGLLLFLVMFIYAIFGMNWFSKVKRGSGIDDIFNFDTFSGSMLCLFQITTSAGWDALLNPMLESKASCNSSSQESCQQPQIAIVYFVSYIIISFLIVVNMYIAVILENFNTATEESEDPLGEDDFEIFYEIWEKFDPEATQFIQYSSLSDFADALPEPLRVAKPNRFQFLMMDLPMVMGDRLHCMDVLFAFTTRVLGNSSGLDTMKAMMEEKFMEANPFKKLYEPIVTTTKRKEEEECAAVIQRAYRRHMEKMIKLKLKGRSSSSLQVFCNGDLSSLDVPKIKVHCD

SEQ ID NO: 7 - DNA, 23 bp, Rattus norvegicus
  misc_feature: Primer sequence
gcaagaaatgcaggaggaaaaac

SEQ ID NO: 8 - DNA, 38 bp, Rattus norvegicus
  misc_feature: Primer sequence
ataagaatgcggccgccaacctgtcacctcgttcagcc

SEQ ID NO: 9 - DNA, 24 bp, Rattus norvegicus
  misc_feature: Primer sequence
gaacaaatgtcaagcctttgtgtt

SEQ ID NO: 10 - DNA, 28 bp, Rattus norvegicus
  misc_feature: Primer sequence
cagccatcatgataatcatatttaagac

SEQ ID NO: 11 - DNA, 16 bp, Artificial Sequence
  Forward strand of SalI linker with NotI/XbaI overhangs
  misc_feature: Forward strand of SalI linker with NotI/XbaI overhangs
ggccgcaaagtcgact

SEQ ID NO: 12 - DNA, 16 bp, Artificial Sequence
  Reverse strand of SalI linker with NotI/XbaI overhangs
  misc_feature: Reverse strand of SalI linker with NotI/XbaI overhangs
cgtttcagctgagatc

SEQ ID NO: 13 - DNA, 21 bp, Rattus norvegicus
  misc_feature: primer sequence
gccttcgtggtcatctttacc

SEQ ID NO: 14 - DNA, 40 bp, Artificial Sequence
  engineers NotI and ClaI sites at 3′ end of rNaN ORF
  misc_feature: primer sequence
aaaagcggccgctttatcgatgtcattgtgaaccttcacc

SEQ ID NO: 15 - DNA, 46 bp, Artificial Sequence
  Forward primer amplifying GFP from pEGFP-C2
  misc_feature: primer sequence
aaaatcgatcatcatcatcatcatcatatggtgagcaagggcgagg

SEQ ID NO: 16 - DNA, 34 bp, Artificial Sequence
  Reverse primer amplifying GFP from pEGFP-C2
  misc_feature: primer sequence
aaaagcggccgcctacttgtacagctcgtccatg

SEQ ID NO: 17 - DNA, 23 bp, Artificial Sequence
  Forward primer
  misc_feature: primer sequence
cagagaccaagcagctcactagc

SEQ ID NO: 18 - DNA, 40 bp, Artificial sequence
  Reverse primer
  misc_feature: primer sequence
aaagcggccgcatcgattatcagtcattgtgaaccttgac

SEQ ID NO: 19 - DNA, 5298 bp, Rattus norvegicus
  CDS (1)..(5298): Corresponds to ORF of Seq ID No. 1 with silent C>T at nt 1264 and T>C at nt 4056
atggaggagaggtactacccggtgatcttcccggacgagcggaatttccgccccttcacttccgactctctggctgccatagagaagcggattgctatccaaaaggagaggaagaagtccaaagacaaggcggcagctgagccccagcctcggcctcagcttgacctaaaggcctccaggaagttacctaagctttatggtgacattccccctgagcttgtagcgaagcctctggaagacctggacccattctacaaagaccataagacattcatggtgttgaacaagaagagaacaatttatcgcttcagcgccaagcgggccttgttcattctggggccttttaatcccctcagaagcttaatgattcgtatctctgtccattcagtctttagcatgttcatcatctgcacggtgatcatcaactgtatgttcatggcgaattctatggagagaagtttcgacaacgacattcccgaatacgtcttcattgggatttatattttagaagctgtgattaaaatattggcaagaggcttcattgtggatgagttttccttcctccgagatccgtggaactggctggacttcattgtcattggaacagcgatcgcaacttgttttccgggcagccaagtcaatctttcagctcttcgtaccttccgagtgttcagagctctgaaggcgatttcagttatctcaggtctgaaggtcatcgtaggtgccctgctgcgctcggtgaagaagctggtagacgtgatggtcctcactctcttctgcctcagcatctttgccctggtcggtcagcagctgttcatgggaattctgaaccagaagtgtattaagcacaactgtggccccaaccctgcatccaacaaggattgctttgaaaaggaaaaagatagcgaagacttcataatgtgtggtacctggctcggcagcagaccctgtcccaatggttctacgtgcgataaaaccacattgaacccagacaataattatacaaagtttgacaactttggctggtcctttctcgccatgttccgggttatgactcaagactcctgggagaggctttaccgacagatcctgcggacctctgggatctactttgtcttcttcttcgtggtggtcatcttcctgggctccttctacctgcttaacctaaccctggctgttgtcaccatggcttatgaagaacagaacagaaatgtagctgctgagacagaggccaaggagaaaatgtttcaggaagcccagcagttgttaagggaggagaaggaggctctggttgccatgggaattgacagaagttcccttaattcccttcaagcttcatccttttccccgaagaagaggaagtttttcggtagtaagacaagaaagtccttctttatgagagggtccaagacggcccaagcctcagcgtctgattcagaggacgatgcctctaaaaatccacagctccttgagcagaccaaacgactgtcccagaacttgccagtggatctctttgatgagcacgtggaccccctccacaggcagagagcgctgagcgctgtcagtatcttaaccatcaccatgcaggaacaagaaaaattccaggagccttgtttcccatgtgggaaaaatttggcctctaagtacctggtgtgggactgtagccctcagtggctgtgcataaagaaggtcctgcggaccatcatgacggatccctttactgagctggccatcaccatctgcatcatcatcaataccgttttcttagccgtggagcaccacaacatggatgacaacttaaagaccatactgaaaataggaaactgggttttcacgggaattttcatagcggaaatgtgtctcaagatcatcgcgctcgacccttaccactacttccggcacggctggaatgtttttgacagcatcgtggccctcctgagtctcgctgatgtgctctacaacacactgtctgataacaataggtctttcttggcttccctcagagtgctgagggtcttcaagttagccaaatcctggcccacgttaaacactctcattaagatcatcggccactccgtgggcgcgcttggaaacctgactgtggtcctgactatcgtggtcttcatcttttctgtggtgggcatgcggctcttcggcaccaagtttaacaagaccgcctacgccacccaggagcggcccaggcggcgctggcacatggataatttctaccactccttcctggtggtgttccgcatcctctgtggggaatggatcgagaacatgtggggctgcatgcaggatatggacggctccccgttgtgcatcattgtctttgtcctgataatggtgatcgggaagcttgtggtgcttaacctcttcattgccttgctgctcaattccttcagcaatgaggagaaggatgggagcctggaaggagagaccaggaaaaccaaagtgcagctagccctggatcggttccgccgggccttctccttcatgctgcacgctcttcagagtttttgttgcaagaaatgcaggaggaaaaactcgccaaagccaaaagagacaacagaaagctttgctggtgagaataaagactcaatcctcccggatgcgaggccctggaaggagtatgatacagacatggctttgtacactggacaggccggggctccgctggccccactcgcagaggtagaggacgatgtggaatattgtggtgaaggcggtgccctacccacctcacaacatagtgctggagttcaggccggtgacctccctccagagaccaagcagctcactagcccggatgaccaaggggttgaaatggaagtattttctgaagaagatctgcatttaagcatacagagtcctcgaaagaagtctgacgcagtgagcatgctctcggaatgcagcacaattgacctgaatgatatctttagaaatttacagaaaacagtttcccccaaaaagcagccagatagatgctttcccaagggccttagttgtcactttctatgccacaaaacagacaagagaaagtccccctgggtcctgtggtggaacattcggaaaacctgctaccaaatcgtgaagcacagctggtttgagagtttcataatctttgttattctgctgagcagtggagcgctgatatttgaagatgtcaatctccccagccggccccaagttgagaaattactaaggtgtaccgataatattttcacatttattttcctcctggaaatgatcctgaagtgggtggcctttggattccggaggtatttcaccagtgcctggtgctggcttgatttcctcattgtggtggtgtctgtgctcagtctcatgaatctaccaagcttgaagtccttccggactctgcgggccctgagacctctgcgggcgctgtcccagtttgaaggaatgaaggttgtcgtctacgccctgatcagcgccatacctgccattctcaatgtcttgctggtctgcctcattttctggctcgtattttgtatcttgggagtaaatttattttctgggaagtttggaaggtgcattaacgggacagacataaatatgtatttggattttaccgaagttccgaaccgaagccaatgtaacattagtaattactcgtggaaggtcccgcaggtcaactttgacaacgtggggaatgcctatctcgccctgctgcaagtggcaacctataagggctggctggaaatcatgaatgctgctgtcgattccagagagaaagacgagcagccggactttgaggcgaacctctacgcgtatctctactttgtggtttttatcatcttcggctccttctttaccctgaacctctttatcggtgttattattgacaacttcaatcagcagcagaaaaagttaggtggccaagacatttttatgacagaagaacagaagaaatattacaatgcaatgaaaaagttaggaaccaagaaacctcaaaagcccatcccaaggcccctgaacaaatgtcaagcctttgtgttcgacctggtcacaagccaggtctttgacgtcatcatcctgggtcttattgtcttaaatatgattatcatgatggctgaatctgccgaccagcccaaagatgtgaagaaaacctttgatatcctcaacatagccttcgtggtcatctttaccatagagtgtctcatcaaagtctttgctttgaggcaacactacttcaccaatggctggaacttatttgattgtgtggtcgtggttctttctatcattagtaccctggtttcccgcttggaggacagtgacatttctttcccgcccacgctcttcagagtcgtccgcttggctcggattggtcgaatcctcaggctggtccgggctgcccggggaatcaggaccctcctctttgctttgatgatgtctctcccctctctcttcaacatcggtctgctgctcttcctggtgatgttcatttacgccatctttgggatgagctggttttccaaagtgaagaagggctccgggatcgacgacatcttcaacttcgagacctttacgggcagcatgctgtgcctcttccagataaccacttcggctggctgggataccctcctcaaccccatgctggaggcaaaagaacactgcaactcctcctcccaagacagctgtcagcagccgcagatagccgtcgtctacttcgtcagttacatcatcatctccttcctcatcgtggtcaacatgtacatcgctgtgatcctcgagaacttcaacacagccacggaggagagcgaggaccctctgggagaggacgactttgaaatcttctatgaggtctgggagaagtttgaccccgaggcgtcgcagttcatccagtattcggccctctctgactttgcggacgccctgccggagccgttgcgtgtggccaagccgaataagtttcagtttctagtgatggacttgcccatggtgatgggcgaccgcctccattgcatggatgttctctttgctttcactaccagggtcctcggggactccagcggcttggataccatgaaaaccatgatggaggagaagtttatggaggccaacccttttaagaagctctacgagcccatagtcaccaccaccaagaggaaggaggaggagcaaggcgccgccgtcatccagagggcctaccggaaacacatggagaagatggtcaaactgaggctgaaggacaggtcaagttcatcgcaccaggtgttttgcaatggagacttgtccagcttggatgtggccaaggtcaaggttcacaatgactga

SEQ ID NO: 20 - protein, 1765 aa, Rattus norvegicus; identical to SEQ ID NO: 2

SEQ ID NO: 21 - protein, 8 aa, Rattus norvegicus
IDHHHHHH

SEQ ID NO: 22 - protein, 16 aa, Artificial Sequence
  Description of Artificial Sequence  Peptide for raising antibodies
CNGDLSSFGVAKGKVH

SEQ ID NO: 23 - protein, 18 aa, Artificial Sequence
  Description of Artificial Sequence  Peptide3 for raising antibodies
CNDGLSSLDVAKVKVHND

SEQ ID NO: 24 - DNA, 32 bp, Artificial Sequence
  Description of Artificial Sequence  PCR primer
acgccttaaggtgaagatggatgacagatgct

SEQ ID NO: 25 - DNA, 30 bp, Artificial Sequence
  Description of Artificial Sequence  PCR primer
ggttctagatcagtcacagtggaccttgcc

SEQ ID NO: 26 - DNA, 23 bp, Artificial Sequence
  Description of Artificial Sequence  PCR primer
gcctcggcgaaagtagtggtagg

SEQ ID NO: 27 - DNA, 22 bp, Artificial Sequence
  Description of Artificial Sequence  PCR primer
cctaccactactttcgccgagg

SEQ ID NO: 28 - DNA, 24 bp, Artificial Sequence
  Description of Artificial Sequence  PCR primer
tacgaagtaaatgtaaccgagtga

SEQ ID NO: 29 - DNA, 23 bp, Artificial Sequence
  Description of Artificial Sequence  PCR primer
ctcggttacatttacttcgtagt

SEQ ID NO: 30 - DNA, 40 bp, Artificial Sequence
  Description of Artificial Sequence  PCR primer
tatatatgcggccgcaagatggaggagaggtactatccag

SEQ ID NO: 31 - DNA, 24 bp, Artificial Sequence
  Description of Artificial Sequence  PCR primer
tattgtggtgttccatggccaaga

SEQ ID NO: 32 - DNA, 21 bp, Artificial Sequence
  Description of Artificial Sequence  PCR primer
gccatggaacaccacaatatg

SEQ ID NO: 33 - DNA, 24 bp, Artificial Sequence
  Description of Artificial Sequence  PCR primer
cacgaagtaaaggtatgcgtatag

SEQ ID NO: 34 - DNA, 24 bp, Artificial Sequence
  Description of Artificial Sequence  PCR primer
tatgaatgcagctgttgattccag

SEQ ID NO: 35 - DNA, 41 bp, Artificial Sequence
  Description of Artificial Sequence  PCR primer
gatcgatcgggccctcagtcacaatgaaccttgatcttggg